(19) United States
(12) Patent Application Publication
(10) Pub. No.: US 2013/0085115 A1
(43) Pub. Date: Apr. 4, 2013

(54) COMBINATION THERAPY FOR LYMPHOMA

(71) Applicant: Celgene Corporation, Summit, NJ (US)

(72) Inventors: Reinhard Dummer, Zurich (CH); Sima Rosati, Zurich (CH)

(73) Assignee: Celgene Corporation, Summit, NJ (US)

(21) Appl. No.: 13/624,807

(22) Filed: Sep. 21, 2012

Related U.S. Application Data
(60) Provisional application No. 61/538,734, filed on Sep. 23, 2011.
(60) Provisional application No. 61/698,441, filed on Sep. 7, 2012.

(51) Int. Cl. A61K 31/706; A61K 31/395

(52) U.S. Cl. CPC A61K 31/706; A61K 31/395
USPC 514/43

(57) ABSTRACT

Methods of treating, preventing or managing lymphomas are disclosed. The methods encompass the administration of an HDAC inhibitor romidepsin and a DNA demethylating agent 5-azacitidine, also known as VIDAZA®. Pharmaceutical compositions and single unit dosage forms suitable for use in the methods provided herein are also disclosed

### CROSS REFERENCE TO RELATED APPLICATIONS

This application claims the benefit of priority to U.S. Provisional Patent Application Ser. No. 61/538,734 filed Sep. 23, 2011 and U.S. Provisional Patent Application Ser. No. 61/698,441 filed Sep. 7, 2012, the disclosures of which are incorporated by reference herein in their entirety.

### FIELD

Provided are methods for treating lymphomas using a combination of a histone deacetylase (HDAC) inhibitor and a DNA demethylating agent. In one embodiment, the HDAC inhibitor is romidepsin. In another embodiment, the DNA demethylating agent is 5-azacitidine. In yet another embodiment, the lymphoma is cutaneous T-cell lymphoma (CTCL).

### BACKGROUND

Lymphoma is a cancer in the lymphatic cells of the immune system. Typically, lymphomas present as a solid tumor of lymphoid cells. These malignant cells often originate in lymph nodes, presenting as an enlargement of the node, i.e., a tumor. It can also affect other organs in which case it is referred to as extranodal lymphoma. Extranodal sites include the skin, brain, bowels and bone. Lymphomas are closely related to lymphoid leukemias, which also originate in lymphocytes but typically involve only circulating blood and the bone marrow and do not usually form static tumors (Parham, P. The immune system. New York: Garland Science. p. 414, 2005). Treatment involves chemotherapy and in some cases radiotherapy and/or bone marrow transplantation, and can be curable depending on the histology, type, and stage of the disease (Parham, P., supra).

Classification of lymphomas is complicated. The most accepted by skilled artisan classification defines lymphomas as mature B-cell lymphomas, mature T-cell and natural killer cell lymphomas, Hodgkin's lymphomas and immunodeficiency-associated lymphoproliferative disorders.

Cutaneous T cell lymphoma (CTCL) is a cancer of mature T cells and is caused by a mutation of these cells. The malignant T cells in the body initially migrate to the skin, causing various lesions to appear. These lesions change shape as the disease progresses, typically beginning as what appears to be a rash which can be very itchy and eventually forming plaques and tumors before metastasizing to other parts of the body.

Tumor cells in CTCL frequently display chromosomal abnormalities (up to 50% of cases) and commonly present a clonal population which is characterized by PCR detectable TCR gene rearrangement (Dummer et al., Arch Dermatol Res 291(6):307-311, 1999; Schwab et al., Br J Haematol 118(4):1019-1026, 2002). Several studies have been carried out in order to characterize chromosomal aberrations; however revealed abnormalities were of moderate reoccurrence (Caprini, et al., Cancer Res 69(21):8438-8446, 2009; Pham-Ledard et al., J Invest Dermatol 130(3):816-825, 2010; Van Doom et al., Blood 113(1):127-136, 2009; Vermeer et al., Cancer Res 68(8):2689-2698, 2008). Studies on high-throughput gene expression profiling of CTCL tumor cells provided useful hints on which molecules may play a role in disease development (Van Doom et al., Cancer Res 64(16):5578-5586, 2004; Booken et al., Leukemia 22(2):393-399, 2008; Mao et al., Blood 101(4):1513-1519, 2003; Mao et al., J Invest Dermatol 126(6):1388-1395, 2006). This observation led to the idea of the role of epigenetic control in the pathogenesis of CTCL.

The pattern of methylation has recently become an important topic for research. Studies have found that in normal tissue methylation of a gene is mainly localized in the coding region, which is cytosine-phosphate-guanine (CpG) poor. In contrast, the promoter region of the gene is unmethylated despite a high density of CpG islands in the region.

Cancer is characterized by “methylation imbalance” where genome-wide hypomethylation is accompanied by localized hypermethylation and an increase in expression of DNA methyltransferase (Chen et al., Nature 395 (6697):89-93, 1998). The overall methylation state in a cell might also be a precipitating factor in carcinogenesis as evidence suggests that genome-wide hypomethylation can lead to chromosome instability and increased mutation rates (Baylin et al., Adv. Cancer Res. 72:141-96, 1998).

The chromatin structure is maintained and regulated through DNA methylation and histone modifications, such as histone acetylation (Eden et al., Nature 394(6696):842, 1998). Methylated DNA stretches attract histone deacetylase which in turn leads to chromatin remodeling and altered gene expression (Jones et al., Nat Genet. 19(2):187-191, 1998; Cameron et al., Nat Genet. 21(1):103-107, 999; Witt et al., Cancer Lett 277(1):8-21, 2009; Marks et al., Adv Cancer Res 91:137-168, 2004). In vitro experiments showed that HDAC inhibitors and demethylating agents work synergistically in terms of regulation (release/suppression) of gene expression. Additionally, clinically promising results have been observed for both agent types as monotherapy in hematopoietic neoplasms (Wu et al., Arch Dermatol 147(4):443-449, 2011).

The current success of HDAC in the clinical practice for CTCL treatment encourages the pursuing of combinational therapy in order to increase the response rate. An effective and safe combinational therapy would be very valuable in a type of cancer where few treatment alternatives exist.

### SUMMARY

In one embodiment, provided herein are methods for treating, preventing or managing lymphoma in a patient comprising administering to said patient an effective amount of an HDAC inhibitor in combination with a DNA demethylating agent.

HDAC inhibitors useful in the methods provided herein include, but are not limited to, trichostatin A (TSA), Vorinostat (SAHA), Valproic Acid (VPA), romidepsin and MS-275. In one embodiment, the HDAC inhibitor is romidepsin.

DNA demethylating agents useful in the methods provided herein include, but are not limited to, 5-azacytidine (azacytidine), 5-azadeoxycytidine (decitabine), zebularine and procaine. In one embodiment, the DNA demethylating agent is 5-azacytidine.

The hematological malignancies treated by the methods provided herein include, but are not limited to, lymphomas, leukemias, multiple myeloma, plasma cell-derived cancers, relapsed hematological malignancies, and refractory hematological malignancies. In one embodiment, lymphomas that can be treated by the methods provided herein include, but are not limited to, mature B-cell lymphomas, mature T-cell and natural killer cell lymphomas, Hodgkin's lymphomas and immunodeficiency-associated lymphoproliferative disorders. In another embodiment, lymphomas that can be treated by the methods provided herein include, but are not limited to, small lymphocytic lymphoma, follicular lymphoma, Mantle cell lymphoma, diffuse large B-cell lymphoma, Burkitt lymphoma, B-cell lymphoblastic lymphoma, small cleaved B-cell lymphoma, non-cleaved B-cell lymphoma, cutaneous T-cell lymphoma (CTCL), and peripheral T-cell lymphoma (PTCL). In one embodiment, lymphoma is T-cell lymphoma. In another embodiment, T-cell lymphoma is cutaneous T-cell lymphoma (CTCL).

In another embodiment, provided herein is a pharmaceutical composition for treating, preventing or managing lymphoma in a patient comprising an HDAC inhibitor and a DNA demethylating agent. In one embodiment, the HDAC inhibitor is romidepsin. In another embodiment, the DNA demethylating agent is 5-azacytidine.

In yet another embodiment, provided herein are single unit dosage forms, dosing regimens and kits which comprise an HDAC inhibitor and a DNA demethylating agent. In one embodiment, the HDAC inhibitor is romidepsin. In another embodiment, the DNA demethylating agent is 5-azacytidine.

### BRIEF DESCRIPTION OF THE DRAWINGS

FIG. 1 depicts the effects of the individual and combined treatment of CTCL cell line with various concentrations of romidepsin and 5-azacitidine on apoptosis (by Annexin V+/PI−). The CTCL cell line SeAx was pretreated with increasing concentrations of 5-azacitidin for 48 hours, then romidepsin was added for 24 hours.

FIGS. 2A and 2B depict the expression levels of p21 and DNMT1 after the treatment of CTCL cell line SeAx with the combination of romidepsin and 5-azacitidine.

FIGS. 3A and 3B depict the induction levels of p21, p15 and the level of acetylation of H3 after the treatment of CTCL cell line SeAx with the combination of romidepsin and 5-azacitidine.

FIGS. 4A and 4B depict the effects of individual and combined treatment of romidepsin and 5-azacitidine on cell viability in MyLa (4A) and SeAx (4B) cell lines.

FIGS. 5A-5D depict the effect of DMSO (5A), 5-azacitidine (5B), romidepsin (5C), and a combination of romidepsin and 5-azacitidine (5D) on cell apoptosis (based on Annexin V assay as measured by flow cytometry).

FIG. 6 depicts the induction levels of β-Actin and DNMT1 and the level of acetylation of H3 based on individual and combined treatment with romidepsin and azacytidine.

FIG. 7 shows the effect of individual and combined treatment with romidepsin and 5-azacytidine on expression of genes responsible for regulation of cell cycle (p21, p15, and β-Actin).

FIGS. 8A-8D show the effect of the treatment with DMSO (8A), 5-azacitidine (8B), romidepsin (8C), and a combination of romidepsin and 5-azacitidine (8D) on the expression of cell cycle regulatory gene p16 based on immunohistochemical assay.

FIG. 9 depicts the effect of the treatment with 5-azacitidine, romidepsin, and their combination on the caspase-cascade apoptosis regulating pathway.

### DETAILED DESCRIPTION

### Definitions

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. It should also be noted that use of “or” means “and/or” unless stated otherwise. Furthermore, use of the term “including” as well as other forms, such as “include,” “includes,” and “included” is not limiting.

The term “treating” as used herein, means an alleviation, in whole or in part, of symptoms associated with a disorder or disease (e.g., cancer or a tumor syndrome), or slowing, or halting of further progression or worsening of those symptoms.

The term “preventing” as used herein, means the prevention of the onset, recurrence or spread, in whole or in part, of the disease or disorder (e.g., cancer), or a symptom thereof.

The term “effective amount” in connection with the HDAC inhibitor means an amount capable of alleviating, in whole or in part, symptoms associated with a disorder, for example cancer, or slowing or halting further progression or worsening of those symptoms, or preventing or providing prophylaxis for cancer, in a subject at risk for cancer. The effective amount of the HDAC inhibitor, for example in a pharmaceutical composition, may be at a level that will exercise the desired effect; for example, about 0.005 mg/kg of a subject's body weight to about 100 mg/kg of a subject's body weight in unit dosage for both oral and parenteral administration. As will be apparent to those skilled in the art, it is to be expected that the effective amount of an HDAC inhibitor disclosed herein may vary depending on the severity of the indication being treated.

The term “pharmaceutically acceptable carrier” as used herein means a pharmaceutically acceptable material, composition or vehicle, such as a liquid or solid filler, diluent, excipient, solvent or encapsulating material, involved in carrying or transporting the subject compounds from the administration site of one organ, or portion of the body, to another organ, or portion of the body, or in an in vitro assay system. Each carrier must be “acceptable” in the sense of being compatible with the other ingredients of the formulation and not injurious to a subject to whom it is administered. Nor should an acceptable carrier alter the specific activity of the subject compounds.

The term “pharmaceutically acceptable” refers to molecular entities and compositions that are physiologically tolerable and do not typically produce an allergic or similar untoward reaction, such as gastric upset, dizziness and the like, when administered to a human.

The term “pharmaceutically acceptable salt” encompasses non-toxic acid and base addition salts of the compound to which the term refers. Acceptable non-toxic acid addition salts include those derived from organic and inorganic acids or bases know in the art, which include, for example, hydrochloric acid, hydrobromic acid, phosphoric acid, sulfuric acid, methanesulphonic acid, acetic acid, tartaric acid, lactic acid, succinic acid, citric acid, malic acid, maleic acid, sorbic acid, aconitic acid, salicylic acid, phthalic acid, embolic acid, enanthic acid, and the like.

Compounds that are acidic in nature are capable of forming salts with various pharmaceutically acceptable bases. The bases that can be used to prepare pharmaceutically acceptable base addition salts of such acidic compounds are those that form non-toxic base addition salts, i.e., salts containing pharmacologically acceptable cations such as, but not limited to, alkali metal or alkaline earth metal salts and the calcium, magnesium, sodium or potassium salts in particular. Suitable organic bases include, but are not limited to, N,N-dibenzylethylenediamine, chloroprocaine, choline, diethanolamine, ethylenediamine, meglumaine (N-methylglucamine), lysine, and procaine.

The term “prodrug” means a derivative of a compound that can hydrolyze, oxidize, or otherwise react under biological conditions (in vitro or in vivo) to provide the compound. Examples of prodrugs include, but are not limited to, derivatives of immunomodulatory compounds of the invention that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include derivatives of immunomodulatory compounds of the invention that comprise —NO, —NO₂, —ONO, or —ONO₂ moieties. Prodrugs can typically be prepared using well-known methods, such as those described in 1 Burger's Medicinal Chemistry and Drug Discovery, 172-178, 949-982 (Manfred E. Wolff ed., 5th ed. 1995), and Design of Prodrugs (H. Bundgaard ed., Elselvier, New York 1985).

The term “unit dose” when used in reference to a therapeutic composition refers to physically discrete units suitable as unitary dosage for humans, each unit containing a predetermined quantity of active material calculated to produce the desired therapeutic effect in association with the required diluent; i.e., carrier, or vehicle.

The term “unit-dosage form” refers to a physically discrete unit suitable for administration to a human and animal subject, and packaged individually as is known in the art. Each unit-dose contains a predetermined quantity of an active ingredient(s) sufficient to produce the desired therapeutic effect, in association with the required pharmaceutical carriers or excipients. A unit-dosage form may be administered in fractions or multiples thereof. Examples of a unit-dosage form include an ampoule, syringe, and individually packaged tablet and capsule.

The term “multiple-dosage form” is a plurality of identical unit-dosage forms packaged in a single container to be administered in segregated unit-dosage form. Examples of a multiple-dosage form include a vial, bottle of tablets or capsules, or bottle of pints or gallons.

The term “tumor” refers to all neoplastic cell growth and proliferation, whether malignant or benign, and all pre-cancerous and cancerous cells and tissues. As used herein, the term “neoplastic” refers to any form of dysregulated or unregulated cell growth, whether malignant or benign, resulting in abnormal tissue growth. Thus, “neoplastic cells” include malignant and benign cells having dysregulated or unregulated cell growth.

The term “cancer” includes, but is not limited to, solid tumors and blood born tumors. The term “cancer” refers to disease of skin tissues, organs, blood, and vessels, including, but not limited to, cancers of the bladder, bone or blood, brain, breast, cervix, chest, colon, endrometrium, esophagus, eye, head, kidney, liver, lymph nodes, lung, mouth, neck, ovaries, pancreas, prostate, rectum, stomach, testis, throat, and uterus.

The term “proliferative” disorder or disease refers to unwanted cell proliferation of one or more subset of cells in a multicellular organism resulting in harm (i.e., discomfort or decreased life expectancy) to the multicellular organism. For example, as used herein, proliferative disorder or disease includes neoplastic disorders and other proliferative disorders.

The term “relapsed” refers to a situation where a subject, that has had a remission of cancer after a therapy, has a return of cancer cells.

The term “refractory” or “resistant” refers to a circumstance where a subject, even after intensive treatment, has residual cancer cells in the body.

The term “lymphoma” means a type of cancer occurred in the lymphatic cells of the immune system and includes, but is not limited to, mature B-cell lymphomas, mature T-cell and natural killer cell lymphomas, Hodgkin's lymphomas and immunodeficiency-associated lymphoproliferative disorders.

The term “cutaneous T-Cell Lymphoma (CTCL)” refers to lymphoma of the skin. It arises from T-cells, is not a single disease, but a group of different lymphomas that affect the skin primarily. These include Mycosis fungoides, Sezary syndrome, Reticulum cell sarcoma of the skin, among others.

The terms “active ingredient” and “active substance” refer to a compound, which is administered, alone or in combination with one or more pharmaceutically acceptable excipients, to a subject for treating, preventing, or ameliorating one or more symptoms of a condition, disorder, or disease. As used herein, “active ingredient” and “active substance” may be an optically active isomer or an isotopic variant of a compound described herein.

The terms “drug,” “therapeutic agent,” and “chemotherapeutic agent” refer to a compound, or a pharmaceutical composition thereof, which is administered to a subject for treating, preventing, or ameliorating one or more symptoms of a condition, disorder, or disease.

The terms “co-administration” and “in combination with” include the administration of two or more therapeutic agents simultaneously, concurrently or sequentially within no specific time limits unless otherwise indicated. In one embodiment, the agents are present in the cell or in the subject's body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, a first agent can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), essentially concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent.

As used herein, and unless otherwise specified, the terms “composition,” “formulation,” and “dosage form” are intended to encompass products comprising the specified ingredient(s) (in the specified amounts, if indicated), as well as any product(s) which result, directly or indirectly, from combination of the specified ingredient(s) in the specified amount(s).

A cytidine analog referred to herein is intended to encompass the free base of the cytidine analog, or a salt, solvate, hydrate, cocrystal, complex, prodrug, precursor, metabolite, and/or derivative thereof. In certain embodiments, a cytidine analog referred to herein encompasses the free base of the cytidine analog, or a salt, solvate, hydrate, cocrystal or complex thereof. In certain embodiments, a cytidine analog referred to herein encompasses the free base of the cytidine analog, or a pharmaceutically acceptable salt, solvate, or hydrate thereof.

The term “hydrate” means a compound provided herein or a salt thereof, which further includes a stoichiometric or non-stoichiometric amount of water bound by non-covalent intermolecular forces.

The term “solvate” means a solvate formed from the association of one or more solvent molecules to a compound provided herein. The term “solvate” includes hydrates (e.g., hemihydrate, monohydrate, dihydrate, trihydrate, tetrahydrate, and the like).

As used herein, and unless otherwise specified, a compound described herein is intended to encompass all possible stereoisomers, unless a particular stereochemistry is specified. Where structural isomers of a compound are interconvertible via a low energy barrier, the compound may exist as a single tautomer or a mixture of tautomers. This can take the form of proton tautomerism; or so-called valence tautomerism in the compound, e.g., that contain an aromatic moiety.

In one embodiment, a compound described herein is intended to encompass isotopically enriched analogs. For example, one or more hydrogen position(s) in a compound may be enriched with deuterium and/or tritium. Other suitable isotopes that may be enriched at particular positions of a compound include, but are not limited, C-13, C-14, N-15, O-17, and/or O-18. In one embodiment, a compound described herein may be enriched at more than one position with isotopes, that are the same or different.

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

Romidepsin

Romidepsin is a natural product which was isolated from Chromobacterium violaceum by Fujisawa Pharmaceuticals (Published Japanese Patent Application No. 64872, U.S. Pat. No. 4,977,138, issued Dec. 11, 1990, Ueda et al., J. Antibiot (Tokyo) 47:301-310, 1994; Nakajima et al., Exp Cell Res 241:126-133, 1998; and WO 02/20817; each of which is incorporated herein by reference. It is a bicyclic peptide consisting of four amino acid residues (D-valine, D-cysteine, dehydrobutyrine, and L-valine) and a novel acid (3-hydroxy-7-mercapto-4-heptenoic acid) containing both amide and ester bonds. In addition to the production from C. violaceum using fermentation, romidepsin can also be prepared by synthetic or semi-synthetic means. The total synthesis of romidepsin reported by Kahn et al. involves 14 steps and yields romidepsin in 18% overall yield (Kahn et al. J. Am. Chem. Soc. 118:7237-7238, 1996).

The chemical name of romidepsin is (1S,4S,7Z,10S,16E,21R)-7-ethylidene-4,2′-bis(1-methylethyl)-2-oxa-12,13-dithia-5,8,20,23-tetrazabicyclo[8.7.6]tricos-16-ene-3,6,9,19,22-pentone. The empirical formula is C₂₄H₃₆N₄O₆S₂. The molecular weight is 540.71. At room temperature, romidepsin is a white powder.

Its structure is shown below (formula I):

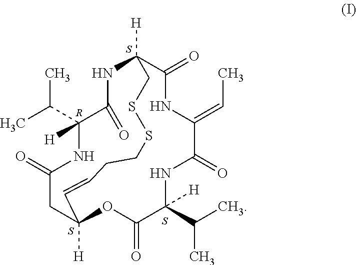

Romidepsin has been shown to have anti-microbial, immunosuppressive, and anti-tumor activities. It was tested, for example, for use in treating patients with hematological malignancies (e.g, cutaneous T-cell lymphoma (CTCL), peripheral T-cell lymphoma (PTCL), multiple myeloma, etc.) and solid tumors (e.g., prostate cancer, pancreatic cancer, etc.) and is thought to act by selectively inhibiting deacetylases (e.g., histone deacetylase, tubulin deacetylase), thus promising new targets for the development of a new class of anti-cancer therapies (Nakajima et al., Exp Cell Res 241:126-133, 1998). One mode of action of romidepsin involves the inhibition of one or more classes of histone deacetylases (HDAC). Preparations and purification of romidepsin is described, for example, in U.S. Pat. No. 4,977,138 and International PCT Application Publication WO 02/20817, each of which is incorporated herein by reference.

Exemplary forms of romidepsin include, but are not limited to, salts, esters, pro-drugs, isomers, stereoisomers (e.g., enantiomers, diastereomers), tautomers, protected forms, reduced forms, oxidized forms, derivatives, and combinations thereof, with the desired activity (e.g., deacetylase inhibitory activity, aggressive inhibition, cytotoxicity). In certain embodiments, romidepsin is a pharmaceutical grade material and meets the standards of the U.S. Pharmacopoeia, Japanese Pharmacopoeia, or European Pharmacopoeia. In certain embodiments, the romidepsin is at least 95%, at least 98%, at least 99%, at least 99.9%, or at least 99.95% pure. In certain embodiments, the romidepsin is at least 95%, at least 98%, at least 99%, at least 99.9%, or at least 99.95% monomeric. In certain embodiments, no impurities are detectable in the romidepsin materials (e.g., oxidized material, reduced material, dimerized or oligomerized material, side products, etc.). Romidepsin typically includes less than 1.0%, less than 0.5%, less than 0.2%, or less than 0.1% of total other unknowns. The purity of romidepsin may be assessed by appearance, HPLC, specific rotation, NMR spectroscopy, IR spectroscopy, UV/Visible spectroscopy, powder x-ray diffraction (XRPD) analysis, elemental analysis, LC-mass spectroscopy, or mass spectroscopy.

Romidepsin is sold under the tradename Istodax® and is approved for the treatment of cutaneous T-cell lymphoma (CTCL) in patients who have received at least one prior systemic therapy, and for the treatment of peripheral T-cell lymphoma (PTCL) in patients who have received at least one prior therapy.

DNA Demethylating Agents

In one embodiment, the methods provided herein comprise administration or co-administration of one or more DNA demethylating agents. In one embodiment, the DNA demethylating agents are cytidine analogs. In certain embodiments, the cytidine analog is 5-azacytidine (5-azacitidine). In certain embodiments, the cytidine analog is 5-aza-2′-deoxycytidine (decitabine). In certain embodiments, the cytidine analog is 5-azacytidine (5-azacitidine) or 5-aza-2′-deoxycytidine (decitabine). In certain embodiments, the cytidine analog is, for example: 1-β-D-arabinofuranosylcytosine (Cytarabine or ara-C); pseudoiso-cytidine (psi ICR); 5-fluoro-2′-deoxycytidine (FCdR); 2′-deoxy-2′,2′-difluorocytidine (Gemcitabine); 5-aza-2′-deoxy-2′,2′-difluorocytidine; 5-aza-2′-deoxy-2′-fluorocytidine; 1-β-D-ribofuranosyl-2(1H)-pyrimidinone (Zebularine); 2′,3′-dideoxy-5-fluoro-3′-thiacytidine (Emtriva); 2′-cyclocytidine (Ancitabine); 1-β-D-arabinofuranosyl-5-azacytosine (Fazarabine or ara-AC); 6-azacytidine (6-aza-CR); 5,6-dihydro-5-azacytidine (dH-aza-C R); N⁴-pentyloxy-carbonyl-5′-deoxy-5-fluorocytidine (Capecitabine); N⁴-octadecyl-cytarabine; or elaidic acid cytarabine. In certain embodiments, the cytidine analogs provided herein include any compound which is structurally related to cytidine or deoxycytidine and functionally mimics and/or antagonizes the action of cytidine or deoxycytidine.

In certain embodiments, exemplary cytidine analogs have the structures provided below:

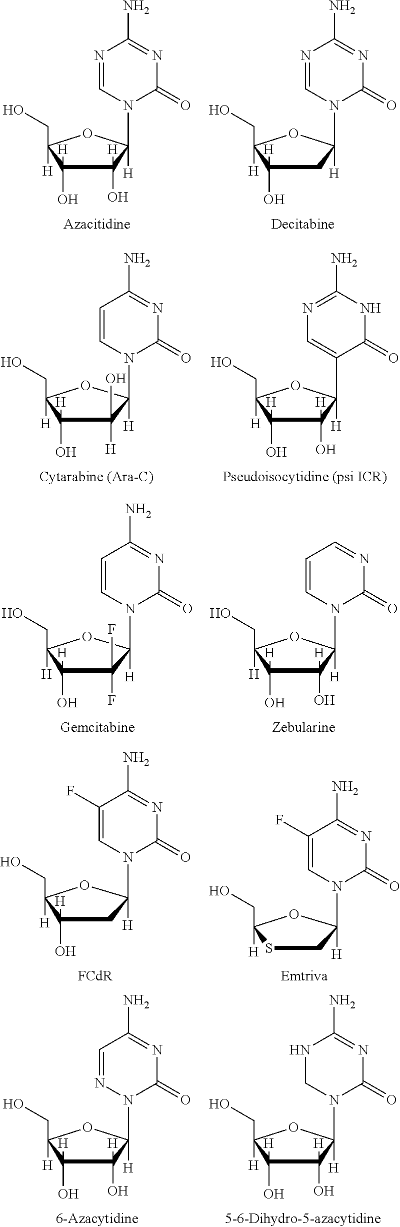

Certain embodiments herein provide salts, cocrystals, solvates (e.g., hydrates), complexes, prodrugs, precursors, metabolites, and/or other derivatives of the cytidine analogs provided herein. For example, particular embodiments provide salts, cocrystals, solvates (e.g., hydrates), complexes, precursors, metabolites, and/or other derivatives of 5-azacytidine. Certain embodiments herein provide salts, cocrystals, and/or solvates (e.g., hydrates) of the cytidine analogs provided herein. Certain embodiments herein provide salts and/or solvates (e.g., hydrates) of the cytidine analogs provided herein. Certain embodiments provide cytidine analogs that are not salts, cocrystals, solvates (e.g., hydrates), or complexes of the cytidine analogs provided herein. For example, particular embodiments provide 5-azacytidine in a non-ionized, non-solvated (e.g., anhydrous), non-complexed form. Certain embodiments herein provide a mixture of two or more cytidine analogs provided herein.

Cytidine analogs provided herein may be prepared using synthetic methods and procedures referenced herein or otherwise available in the literature. For example, particular methods for synthesizing 5-azacytidine are disclosed, e.g., in U.S. Pat. No. 7,038,038 and references discussed therein, each of which is incorporated herein by reference. Other cytidine analogs provided herein may be prepared, e.g., using procedures known in the art, or may be purchased from a commercial source. In one embodiment, the cytidine analogs provided herein may be prepared in a particular solid form (e.g., amorphous or crystalline form). See, e.g., U.S. patent application Ser. No. 10/390,578, filed Mar. 17, 2003 and U.S. patent application Ser. No. 10/390,530, filed Mar. 17, 2003, both of which are incorporated herein by reference in their entireties.

In one embodiment, the compound used in the methods provided herein is a free base, or a pharmaceutically acceptable salt or solvate thereof. In one embodiment, the free base or the pharmaceutically acceptable salt or solvate is a solid. In another embodiment, the free base or the pharmaceutically acceptable salt or solvate is a solid in an amorphous form. In yet another embodiment, the free base or the pharmaceutically acceptable salt or solvate is a solid in a crystalline form. For example, particular embodiments provide 5-azacytidine in solid forms, which can be prepared, for example, according to the methods described in U.S. Pat. Nos. 6,943,249, 6,887,855 and 7,078,518, and U.S. Patent Application Publication Nos. 2005/027675 and 2006/247189, each of which is incorporated by reference herein in their entireties. In other embodiments, 5-azacytidine in solid forms can be prepared using other methods known in the art.

In one embodiment, the compound used in the methods provided herein is a pharmaceutically acceptable salt of the cytidine analog, which includes, but is not limited to, acetate, adipate, alginate, aspartate, benzoate, benzenesulfonate (besylate), bisulfate, butyrate, citrate, camphorate, camphorsulfonate, cyclopentanepropionate, digluconate, dodecylsulfate, 1,2-ethanedisulfonate (edisylate), ethanesulfonate (esylate), formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate (mesylate), 2-naphthalenesulfonate (napsylate), nicotinate, nitrate, oxalate, palmoate, pectinate, persulfate, 3-phenylpropionate, phosphate, picrate, pivalate, propionate, salicylate, succinate, sulfate, tartrate, thiocyanate, tosylate, or undecanoate salts.

Cytidine analogs may be synthesized by methods known in the art. In one embodiment, methods of synthesis include methods as disclosed in U.S. Pat. No. 7,038,038; U.S. Pat. No. 6,887,855; U.S. Pat. No. 7,078,518; U.S. Pat. No. 6,943,249; and U.S. Ser. No. 10/823,394, all incorporated by reference herein in their entireties.

5-azacitidine is 4-amino-1-β-D-ribofuranozyl-s-triazin-2(1H)-one, also known as VIDAZA®. Its empirical formula is C₈H₁₂N₄O₅, the molecular weight is 244. 5-azacitidine is a white to off-white solid that is insoluble in acetone, ethanol and methyl ketone; slightly soluble in ethanol/water (50/50), propylene glycol and polyethylene glycol; sparingly soluble in water, water-saturated octanol, 5% dextrose in water, N-methyl-2-pyrrolidone, normal saline and 5% Tween 80 in water, and soluble in dimethylsulfoxide (DMSO).

VIDAZA® is approved for treatment in patients with higher-risk MDS. It is supplied in a sterile form for reconstitution as a suspension for subcutaneous injection or reconstitution as a solution with further dilution for intravenous infusion. Vials of VIDAZA® contain 100 mg of 5-azacitidine and 100 mg of mannitol as a sterile lyophilized powder.

### Methods of Use

In one embodiment, provided is a method for treating, preventing, or managing lymphoma in a patient comprising administering to said patient an effective amount of HDAC inhibitor in combination with a DNA demethylating agent or a pharmaceutically acceptable salt, solvate, hydrate, stereoisomer, clathrate, or prodrug thereof.

HDAC inhibitors for use in the methods provided herein include, but are not limited to, trichostatin A (TSA), Vorinostat (SAHA), Valproic Acid (VPA), romidepsin and MS-275. In one embodiment, the HDAC inhibitor is romidepsin.

The DNA demethylating agents useful in the methods provided herein include, but are not limited to, 5-azacytidine (azacytidine), 5-azadeoxycytidine (decitabine), zebularine and procaine. In one embodiment, the DNA demethylating agent is 5-azacytidine.

In one embodiment, haematological malignancies that can be treated by the methods provided herein include, but are not limited to, lymphomas, leukemias, multiple myeloma, plasma cell-derived cancers, relapsed hematological malignancies, and refractory hematological malignancies. In one embodiment, lymphomas that can be treated by the methods provided herein include, but are not limited to, mature B-cell lymphomas, mature T-cell and natural killer cell lymphomas, Hodgkin's lymphomas and immunodeficiency-associated lymphoproliferative disorders. In another embodiment, lymphomas that can be treated by the methods provided herein include, but are not limited to, small lymphocytic lymphoma, follicular lymphoma, Mantle cell lymphoma, diffuse large B-cell lymphoma, Burkitt lymphoma, B-cell lymphoblastic lymphoma, small cleaved B-cell lymphoma, non-cleaved B-cell lymphoma, cutaneous T-cell lymphoma (CTCL), and peripheral T-cell lymphoma (PTCL). In one embodiment, lymphoma is T-cell lymphoma. In another embodiment, T-cell lymphoma is cutaneous T-cell lymphoma (CTCL).

Administration of romidepsin and 5-azacytidine can occur simultaneously or sequentially by the same or different routes of administration. The suitability of a particular route of administration employed for a particular active agent will depend on the active agent itself (e.g., whether it can be administered orally without decomposing prior to entering the blood stream) and the disease being treated.

Suitable routes of administration include, but are not limited to, oral, mucosal (e.g., nasal, sublingual, vaginal, buccal, or rectal), parenteral (e.g., subcutaneous, intravenous, bolus injection, intramuscular, or intraarterial), topical (e.g., eye drops or other ophthalmic preparations), transdermal or transcutaneous administration to a patient.

In one embodiment, an effective amount of romidepsin or 5-azacitidine to be used is a therapeutically effective amount. In one embodiment, the amounts of romidepsin or 5-azacitidine to be used in the methods provided herein include an amount sufficient to cause improvement in at least a subset of patients with respect to symptoms, overall course of disease, or other parameters known in the art. Precise amounts for therapeutically effective amounts of romidepsin or 5-azacitidine in the pharmaceutical compositions will vary depending on the age, weight, disease, and condition of the patient.

In one embodiment, romidepsin is administered intravenously. In one embodiment, romidepsin is administered intravenously over a 1-6 hour period. In one embodiment, romidepsin is administered intravenously over a 3-4 hour period. In one embodiment, romidepsin is administered intravenously over a 5-6 hour period. In one embodiment, romidepsin is administered intravenously over a 4 hour period.

In one embodiment, romidepsin is administered in a dose ranging from 0.5 mg/m² to 28 mg/m². In one embodiment, romidepsin is administered in a dose ranging from 0.5 mg/m² to 5 mg/m². In one embodiment, romidepsin is administered in a dose ranging from 1 mg/m² to 25 mg/m². In one embodiment, romidepsin is administered in a dose ranging from 1 mg/m² to 20 mg/m². In one embodiment, romidepsin is administered in a dose ranging from 1 mg/m² to 15 mg/m². In one embodiment, romidepsin is administered in a dose ranging from 2 mg/m² to 15 mg/m². In one embodiment, romidepsin is administered in a dose ranging from 2 mg/m² to 12 mg/m². In one embodiment, romidepsin is administered in a dose ranging from 4 mg/m² to 12 mg/m². In one embodiment, romidepsin is administered in a dose ranging from 6 mg/m² to 12 mg/m². In one embodiment, romidepsin is administered in a dose ranging from 8 mg/m² to 12 mg/m². In one embodiment, romidepsin is administered in a dose ranging from 8 mg/m² to 10 mg/m². In one embodiment, romidepsin is administered in a dose of about 8 mg/m². In one embodiment, romidepsin is administered in a dose of about 9 mg/m². In one embodiment, romidepsin is administered in a dose of about 10 mg/m². In one embodiment, romidepsin is administered in a dose of about 11 mg/m². In one embodiment, romidepsin is administered in a dose of about 12 mg/m². In one embodiment, romidepsin is administered in a dose of about 13 mg/m². In one embodiment, romidepsin is administered in a dose of about 14 mg/m². In one embodiment, romidepsin is administered in a dose of about 15 mg/m².

In one embodiment, romidepsin is administered in a dose of 14 mg/m² over a 4 hour iv infusion on days 1, 8 and 15 of the 28 day cycle. In one embodiment, the cycle is repeated every 28 days.

In one embodiment, increasing doses of romidepsin are administered over the course of a cycle. In one embodiment, the dose of about 8 mg/m² followed by a dose of about 10 mg/m², followed by a dose of about 12 mg/m² is administered over a cycle.

In one embodiment, romidepsin is administered orally. In one embodiment, romidepsin is administered in a dose ranging from 10 mg/m² to 300 mg/m². In one embodiment, romidepsin is administered in a dose ranging from 15 mg/m² to 250 mg/m². In one embodiment, romidepsin is administered in a dose ranging from 20 mg/m² to 200 mg/m². In one embodiment, romidepsin is administered in a dose ranging from 25 mg/m² to 150 mg/m². In one embodiment, romidepsin is administered in a dose ranging from 25 mg/m² to 100 mg/m². In one embodiment, romidepsin is administered in a dose ranging from 25 mg/m² to 75 mg/m².

In one embodiment, romidepsin is administered orally on a daily basis. In one embodiment, romidepsin is administered orally every other day. In one embodiment, romidepsin is administered orally every third, fourth, fifth, or sixth day. In one embodiment, romidepsin is administered orally every week. In one embodiment, romidepsin is administered orally every other week.

In one embodiment, 5-azacitidine is administered by, e.g., intravenous (IV), subcutaneous (SC) or oral routes. Certain embodiments herein provide co-administration of 5-azacytidine with one or more additional active agents to provide a synergistic therapeutic effect in subjects in need thereof. The co-administered agent(s) may be a cancer therapeutic agent, as described herein. In certain embodiments, the co-administered agent(s) may be dosed, e.g., orally or by injection (e.g., IV or SC).

Certain embodiments herein provide methods for treating lymphoma comprising administering 5-azacytidine using, e.g., IV, SC and/or oral administration methods. In certain embodiments, treatment cycles comprise multiple doses administered to a subject in need thereof over multiple days (e.g., 1, 2, 3, 4, 5, 6, 7, 8, 9, 10, 11, 12, 13, 14, or greater than 14 days), optionally followed by treatment dosing holidays (e.g., 1, 2, 3, 4, 5, 6, 7, 8, 9, 10, 11, 12, 13, 14, 15, 16, 17, 18, 19, 20, 21, 22, 23, 24, 25, 26, 27, 28, or greater than 28 days). Suitable dosage amounts for the methods provided herein include, e.g., therapeutically effective amounts and prophylactically effective amounts. For example, in certain embodiments, the amount of 5-azacytidine administered in the methods provided herein may range, e.g., between about 50 mg/m²/day and about 2,000 mg/m²/day, between about 100 mg/m²/day and about 1,000 mg/m²/day, between about 100 mg/m²/day and about 500 mg/m²/day, between about 50 mg/m²/day and about 500 mg/m²/day, between about 50 mg/m²/day and about 200 mg/m²/day, between about 50 mg/m²/day and about 100 mg/m²/day, between about 50 mg/m²/day and about 75 mg/m²/day, or between about 120 mg/m²/day and about 250 mg/m²/day. In certain embodiments, particular dosages are, e.g., about 50 mg/m²/day, about 60 mg/m²/day, about 75 mg/m²/day, about 80 mg/m²/day, about 100 mg/m²/day, about 120 mg/m²/day, about 140 mg/m²/day, about 150 mg/m²/day, about 180 mg/m²/day, about 200 mg/m²/day, about 220 mg/m²/day, about 240 mg/m²/day, about 250 mg/m²/day, about 260 mg/m²/day, about 280 mg/m²/day, about 300 mg/m²/day, about 320 mg/m²/day, about 350 mg/m²/day, about 380 mg/m²/day, about 400 mg/m²/day, about 450 mg/m²/day, or about 500 mg/m²/day. In certain embodiments, particular dosages are, e.g., up to about 100 mg/m²/day, up to about 120 mg/m²/day, up to about 140 mg/m²/day, up to about 150 mg/m²/day, up to about 180 mg/m²/day, up to about 200 mg/m²/day, up to about 220 mg/m²/day, up to about 240 mg/m²/day, up to about 250 mg/m²/day, up to about 260 mg/m²/day, up to about 280 mg/m²/day, up to about 300 mg/m²/day, up to about 320 mg/m²/day, up to about 350 mg/m²/day, up to about 380 mg/m²/day, up to about 400 mg/m²/day, up to about 450 mg/m²/day, up to about 500 mg/m²/day, up to about 750 mg/m²/day, or up to about 1000 mg/m²/day.

In one embodiment, the amount of 5-azacytidine administered in the methods provided herein may range, e.g., between about 5 mg/day and about 2,000 mg/day, between about 10 mg/day and about 2,000 mg/day, between about 20 mg/day and about 2,000 mg/day, between about 50 mg/day and about 1,000 mg/day, between about 100 mg/day and about 1,000 mg/day, between about 100 mg/day and about 500 mg/day, between about 150 mg/day and about 500 mg/day, or between about 150 mg/day and about 250 mg/day. In certain embodiments, particular dosages are, e.g., about 10 mg/day, about 20 mg/day, about 50 mg/day, about 75 mg/day, about 100 mg/day, about 120 mg/day, about 150 mg/day, about 200 mg/day, about 250 mg/day, about 300 mg/day, about 350 mg/day, about 400 mg/day, about 450 mg/day, about 500 mg/day, about 600 mg/day, about 700 mg/day, about 800 mg/day, about 900 mg/day, about 1,000 mg/day, about 1,200 mg/day, or about 1,500 mg/day. In certain embodiments, particular dosages are, e.g., up to about 10 mg/day, up to about 20 mg/day, up to about 50 mg/day, up to about 75 mg/day, up to about 100 mg/day, up to about 120 mg/day, up to about 150 mg/day, up to about 200 mg/day, up to about 250 mg/day, up to about 300 mg/day, up to about 350 mg/day, up to about 400 mg/day, up to about 450 mg/day, up to about 500 mg/day, up to about 600 mg/day, up to about 700 mg/day, up to about 800 mg/day, up to about 900 mg/day, up to about 1,000 mg/day, up to about 1,200 mg/day, or up to about 1,500 mg/day.

In one embodiment, the amount of 5-azacytidine in the pharmaceutical composition or dosage form provided herein may range, e.g., between about 5 mg and about 2,000 mg, between about 10 mg and about 2,000 mg, between about 20 mg and about 2,000 mg, between about 50 mg and about 1,000 mg, between about 50 mg and about 500 mg, between about 50 mg and about 250 mg, between about 100 mg and about 500 mg, between about 150 mg and about 500 mg, or between about 150 mg and about 250 mg. In certain embodiments, particular amounts are, e.g., about 10 mg, about 20 mg, about 50 mg, about 75 mg, about 100 mg, about 120 mg, about 150 mg, about 200 mg, about 250 mg, about 300 mg, about 350 mg, about 400 mg, about 450 mg, about 500 mg, about 600 mg, about 700 mg, about 800 mg, about 900 mg, about 1,000 mg, about 1,200 mg, or about 1,500 mg. In certain embodiments, particular amounts are, e.g., up to about 10 mg, up to about 20 mg, up to about 50 mg, up to about 75 mg, up to about 100 mg, up to about 120 mg, up to about 150 mg, up to about 200 mg, up to about 250 mg, up to about 300 mg, up to about 350 mg, up to about 400 mg, up to about 450 mg, up to about 500 mg, up to about 600 mg, up to about 700 mg, up to about 800 mg, up to about 900 mg, up to about 1,000 mg, up to about 1,200 mg, or up to about 1,500 mg.

In one embodiment, depending on the disease to be treated and the subject's condition, 5-azacytidine may be administered by oral, parenteral (e.g., intramuscular, intraperitoneal, intravenous, CIV, intracistemal injection or infusion, subcutaneous injection, or implant), inhalation, nasal, vaginal, rectal, sublingual, or topical (e.g., transdermal or local) routes of administration. 5-azacytidine may be formulated, alone or together with one or more active agent(s), in suitable dosage unit with pharmaceutically acceptable excipients, carriers, adjuvants and vehicles, appropriate for each route of administration. In one embodiment, 5-azacytidine is administered orally. In another embodiment, 5-azacytidine is administered parenterally. In yet another embodiment, 5-azacytidine is administered intravenously.

In one embodiment, 5-azacytidine can be delivered as a single dose such as, e.g., a single bolus injection, or oral tablets or pills; or over time such as, e.g., continuous infusion over time or divided bolus doses over time. In one embodiment, 5-azacytidine can be administered repetitively if necessary, for example, until the patient experiences stable disease or regression, or until the patient experiences disease progression or unacceptable toxicity. For example, stable disease for solid tumors generally means that the perpendicular diameter of measurable lesions has not increased by 25% or more from the last measurement. See, e.g., Response Evaluation Criteria in Solid Tumors (RECIST) Guidelines, Journal of the National Cancer Institute 92(3): 205-216 (2000). Stable disease or lack thereof is determined by methods known in the art such as evaluation of patient's symptoms, physical examination, visualization of the tumor that has been imaged using X-ray, CAT, PET, or MRI scan and other commonly accepted evaluation modalities.

In one embodiment, 5-azacytidine can be administered once daily or divided into multiple daily doses such as twice daily, three times daily, and four times daily. In one embodiment, the administration can be continuous (i.e., daily for consecutive days or every day), intermittent, e.g., in cycles (i.e., including days, weeks, or months of rest when no drug is administered). In one embodiment, 5-azacytidine is administered daily, for example, once or more than once each day for a period of time. In one embodiment, 5-azacytidine is administered daily for an uninterrupted period of at least 7 days, in some embodiments, up to 52 weeks. In one embodiment, 5-azacytidine is administered intermittently, i.e., stopping and starting at either regular or irregular intervals. In one embodiment, 5-azacytidine is administered for one to six days per week. In one embodiment, 5-azacytidine is administered in cycles (e.g., daily administration for two to eight consecutive weeks, then a rest period with no administration for up to one week; or e.g., daily administration for one week, then a rest period with no administration for up to three weeks). In one embodiment, 5-azacytidine is administered on alternate days. In one embodiment, 5-azacytidine is administered in cycles (e.g., administered daily or continuously for a certain period interrupted with a rest period).

In one embodiment, the frequency of administration ranges from about daily to about monthly. In certain embodiments, 5-azacytidine is administered once a day, twice a day, three times a day, four times a day, once every other day, twice a week, once every week, once every two weeks, once every three weeks, or once every four weeks. In one embodiment, 5-azacytidine is administered once a day. In another embodiment, 5-azacytidine is administered twice a day. In yet another embodiment, 5-azacytidine is administered three times a day. In still another embodiment, 5-azacytidine) is administered four times a day.

In one embodiment, 5-azacytidine is administered once per day from one day to six months, from one week to three months, from one week to four weeks, from one week to three weeks, or from one week to two weeks. In certain embodiments, 5-azacytidine is administered once per day for one week, two weeks, three weeks, or four weeks. In one embodiment, 5-azacytidine is administered once per day for one week. In another embodiment, 5-azacytidine is administered once per day for two weeks. In yet another embodiment, 5-azacytidine is administered once per day for three weeks. In still another embodiment, 5-azacytidine is administered once per day for four weeks.

In one embodiment, 5-azacytidine is administered once per day for about 1 week, about 2 weeks, about 3 weeks, about 4 weeks, about 6 weeks, about 9 weeks, about 12 weeks, about 15 weeks, about 18 weeks, about 21 weeks, or about 26 weeks. In certain embodiments, 5-azacytidine is administered intermittently. In certain embodiments, 5-azacytidine is administered intermittently in the amount of between about 50 mg/m²/day and about 2,000 mg/m²/day. In certain embodiments, 5-azacytidine is administered continuously. In certain embodiments, 5-azacytidine is administered continuously in the amount of between about 50 mg/m²/day and about 1,000 mg/m²/day.

In certain embodiments, 5-azacytidine is administered to a patient in cycles (e.g., daily administration for one week, then a rest period with no administration for up to three weeks). Cycling therapy involves the administration of an active agent for a period of time, followed by a rest for a period of time, and repeating this sequential administration. Cycling therapy can reduce the development of resistance, avoid or reduce the side effects, and/or improves the efficacy of the treatment.

In one embodiment, 5-azacytidine is administered to a patient in cycles. In one embodiment, a method provided herein comprises administering 5-azacytidine in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or greater than 40 cycles. In one embodiment, the median number of cycles administered in a group of patients is about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or greater than about 30 cycles.

In one embodiment, 5-azacytidine is administered to a patient at a dose provided herein over a cycle of 28 days which consists of a 7-day treatment period and a 21-day resting period. In one embodiment, 5-azacytidine is administered to a patient at a dose provided herein each day from day 1 to day 7, followed with a resting period from day 8 to day 28 with no administration of 5-azacytidine. In one embodiment, 5-azacytidine is administered to a patient in cycles, each cycle consisting of a 7-day treatment period followed with a 21-day resting period. In particular embodiments, 5-azacytidine is administered to a patient at a dose of about 50, about 60, about 70, about 75, about 80, about 90, or about 100 mg/m²/d, for 7 days, followed with a resting period of 21 days. In one embodiment, 5-azacytidine is administered intravenously. In one embodiment, 5-azacytidine is administered subcutaneously.

In other embodiments, 5-azacytidine is administered orally in cycles.

Accordingly, in one embodiment, 5-azacytidine is administered daily in single or divided doses for about one week, about two weeks, about three weeks, about four weeks, about five weeks, about six weeks, about eight weeks, about ten weeks, about fifteen weeks, or about twenty weeks, followed by a rest period of about 1 day to about ten weeks. In one embodiment, the methods provided herein contemplate cycling treatments of about one week, about two weeks, about three weeks, about four weeks, about five weeks, about six weeks, about eight weeks, about ten weeks, about fifteen weeks, or about twenty weeks. In some embodiments, 5-azacytidine is administered daily in single or divided doses for about one week, about two weeks, about three weeks, about four weeks, about five weeks, or about six weeks with a rest period of about 1, 3, 5, 7, 9, 12, 14, 16, 18, 20, 22, 24, 26, 28, 29, or 30 days. In some embodiments, the rest period is 1 day. In some embodiments, the rest period is 3 days. In some embodiments, the rest period is 7 days. In some embodiments, the rest period is 14 days. In some embodiments, the rest period is 28 days. The frequency, number and length of dosing cycles can be increased or decreased.

In one embodiment, the methods provided herein comprise: i) administering to the subject a first daily dose of 5-azacytidine; ii) optionally resting for a period of at least one day where 5-azacytidine is not administered to the subject; iii) administering a second dose of 5-azacytidine to the subject; and iv) repeating steps ii) to iii) a plurality of times. In certain embodiments, the first daily dose is between about 50 mg/m²/day and about 2,000 mg/m²/day. In certain embodiments, the second daily dose is between about 50 mg/m²/day and about 2,000 mg/m²/day. In certain embodiments, the first daily dose is higher than the second daily dose. In certain embodiments, the second daily dose is higher than the first daily dose. In one embodiment, the rest period is 2 days, 3 days, 5 days, 7 days, 10 days, 12 days, 13 days, 14 days, 15 days, 17 days, 21 days, or 28 days. In one embodiment, the rest period is at least 2 days and steps ii) through iii) are repeated at least three times. In one embodiment, the rest period is at least 2 days and steps ii) through iii) are repeated at least five times. In one embodiment, the rest period is at least 3 days and steps ii) through iii) are repeated at least three times. In one embodiment, the rest period is at least 3 days and steps ii) through iii) are repeated at least five times. In one embodiment, the rest period is at least 7 days and steps ii) through iii) are repeated at least three times. In one embodiment, the rest period is at least 7 days and steps ii) through iii) are repeated at least five times. In one embodiment, the rest period is at least 14 days and steps ii) through iii) are repeated at least three times. In one embodiment, the rest period is at least 14 days and steps ii) through iii) are repeated at least five times. In one embodiment, the rest period is at least 21 days and steps ii) through iii) are repeated at least three times. In one embodiment, the rest period is at least 21 days and steps ii) through iii) are repeated at least five times. In one embodiment, the rest period is at least 28 days and steps ii) through iii) are repeated at least three times. In one embodiment, the rest period is at least 28 days and steps ii) through iii) are repeated at least five times. In one embodiment, the methods provided herein comprise: i) administering to the subject a first daily dose of 5-azacytidine for 1, 2, 3, 4, 5, 6, 7, 8, 9, 10, 11, 12, 13, or 14 days; ii) resting for a period of 1, 2, 3, 4, 5, 6, 7, 8, 9, 10, 11, 12, 13, 14, 15, 16, 17, 18, 19, 20, 21, 22, 23, 24, 25, 26, 27, or 28 days; iii) administering to the subject a second daily dose of 5-azacytidine for 1, 2, 3, 4, 5, 6, 7, 8, 9, 10, 11, 12, 13, or 14 days; and iv) repeating steps ii) to iii) a plurality of times. In one embodiment, the methods provided herein comprise: i) administering to the subject a daily dose of 5-azacytidine for 1, 2, 3, 4, 5, 6, 7, 8, 9, 10, 11, 12, 13, or 14 days; ii) resting for a period of 1, 2, 3, 4, 5, 6, 7, 8, 9, 10, 11, 12, 13, 14, 15, 16, 17, 18, 19, 20, 21, 22, 23, 24, 25, 26, 27, or 28 days; and iii) repeating steps i) to ii) a plurality of times. In one embodiment, the methods provided herein comprise: i) administering to the subject a daily dose of 5-azacytidine for 7 days; ii) resting for a period of 21 days; and iii) repeating steps i) to ii) a plurality of times. In one embodiment, the daily dose is between about 50 mg/m²/day and about 2,000 mg/m²/day. In one embodiment, the daily dose is between about 50 mg/m²/day and about 1,000 mg/m²/day. In one embodiment, the daily dose is between about 50 mg/m²/day and about 500 mg/m²/day. In one embodiment, the daily dose is between about 50 mg/m²/day and about 200 mg/m²/day. In one embodiment, the daily dose is between about 50 mg/m²/day and about 100 mg/m²/day.

In certain embodiments, 5-azacytidine is administered continuously for between about 1 and about 52 weeks. In certain embodiments, 5-azacytidine is administered continuously for about 1, 2, 3, 4, 5, 6, 7, 8, 9, 10, 11, or 12 months. In certain embodiments, 5-azacytidine is administered continuously for about 14, about 28, about 42, about 84, or about 112 days. It is understood that the duration of the treatment may vary with the age, weight, and condition of the subject being treated, and may be determined empirically using known testing protocols or according to the professional judgment of the person providing or supervising the treatment. The skilled clinician will be able to readily determine, without undue experimentation, an effective drug dose and treatment duration, for treating an individual subject having a particular type of cancer.

In one embodiment, pharmaceutical compositions may contain sufficient quantities of 5-azacytidine to provide a daily dosage of about 10 to 150 mg/m² (based on patient body surface area) or about 0.1 to 4 mg/kg (based on patient body weight) as single or divided (2-3) daily doses. In one embodiment, dosage is provided via a seven-day administration of 75 mg/m² subcutaneously, once every twenty-eight days, for as long as clinically necessary. In one embodiment, dosage is provided via a seven-day administration of 100 mg/m² subcutaneously, once every twenty-eight days, for as long as clinically necessary. In one embodiment, up to 4, up to 5, up to 6, up to 7, up to 8, up to 9 or more 28-day cycles are administered. Other methods for providing an effective amount of 5-azacytidine are disclosed in, for example, “Colon-Targeted Oral Formulations of Cytidine Analogs”, U.S. Ser. No. 11/849,958, and “Oral Formulations of Cytidine Analogs and Methods of Use Thereof”, U.S. Ser. No. 12/466,213, both of which are incorporated by reference herein in their entireties.

In particular embodiments, the number of cycles administered is, e.g., at least 4, at least 5, at least 6, at least 7, at least 8, at least 9, at least 10, at least 11, at least 12, at least 13, at least 14, at least 15, at least 16, at least 17, at least 18, at least 19, at least 20, at least 22, at least 24, at least 26, at least 28, at least 30, at least 32, at least 34, at least 36, at least 38, at least 40, at least 42, at least 44, at least 46, at least 48, or at least 50 cycles of 5-azacitidine treatment. In particular embodiments, the treatment is administered, e.g., 1, 2, 3, 4, 5, 6, 7, 8, 9, 10, 11, 12, 13, or 14 days out of a 28-day period. In particular embodiments, the 5-azacytidine dose is, e.g., at least 10 mg/day, at least 20 mg/day, at least 30 mg/day, at least 40 mg/day, at least 50 mg/day, at least 55 mg/day, at least 60 mg/day, at least 65 mg/day, at least 70 mg/day, at least 75 mg/day, at least 80 mg/day, at least 85 mg/day, at least 90 mg/day, at least 95 mg/day, or at least 100 mg/day.

In particular embodiments, the dosing is performed, e.g., subcutaneously or intravenously. In particular embodiments, the contemplated specific 5-azacytidine dose is, e.g., at least 50 mg/m²/day, at least 60 mg/m²/day, at least 70 mg/m²/day, at least 75 mg/m²/day, at least 80 mg/m2/day, at least 90 mg/m²/day, or at least 100 mg/m²/day. One particular embodiment herein provides administering the treatment for 7 days out of each 28-day period. One particular embodiment herein provides a dosing regimen of 75 mg/m² subcutaneously or intravenously, daily for 7 days. One particular embodiment herein provides a dosing regimen of 100 mg/m² subcutaneously or intravenously, daily for 7 days.

In one embodiment, romidepsin and 5-azacitidine are administered intravenously. In one embodiment, the combination is administered intravenously over a 1-6 hour period. In one embodiment, the combination is administered intravenously over a 3-4 hour period. In one embodiment, the combination is administered intravenously over a 5-6 hour period. In one embodiment, the combination is administered intravenously over a 4 hour period.

In one embodiment, the combination with increasing doses of romidepsin is administered over the course of a cycle. In one embodiment, the dose of about 8 mg/m² followed by a dose of about 10 mg/m², followed by a dose of about 12 mg/m² of romidepsin is administered over a cycle.

In one embodiment, romidepsin is administered intravenously and 5-azacitidine is administered subcutaneously. In one embodiment, romidepsin is administered intravenously and 5-azacitidine is administered orally. In one embodiment, romidepsin and 5-azacitidine are administered orally.

In one embodiment, 5-azacitidine is administered daily based on 7 to 14 days administration every 28-day cycle in a single or divided doses in a four to forty week period with a rest period of about a week or two weeks.

In one embodiment, 5-azacitidine is administered daily and continuously for four to forty weeks at a dose of from about 10 to about 150 mg/m² followed by a break of one or two weeks. In a particular embodiment, 5-azacitidine is administered in an amount of from about 0.1 to about 4.0 mg/day for four to forty weeks, with one week or two weeks of rest in a four or six week cycle.

In one embodiment, 5-azacitidine is administered intravenously to patients with lymphoma in an amount of from about 0.1 to about 4.0 mg per day for about 7 to about 14 days followed by about 14 to about 21 days of rest in a 28 day cycle combined with romidepsin administered intravenously in a dose of about 0.5 mg/m² to about 28 mg/m² administered on days 1, 8 and 15 of the 28 day cycle.

In one embodiment, 5-azacitidine is administered intravenously to patients with lymphoma in an amount of from about 0.10 to about 4.0 mg per day for about 7 to about 14 days followed by about 14 to about 21 day of rest in a 28 day cycle combined with romidepsin administered orally in a dose of about 10 mg/m² to about 300 mg/m² administered on days 1, 8 and 15 of the 28 day cycle.

In one embodiment, 5-azacitidine is administered subcutaneously to patients with lymphoma in an amount of from about 0.10 to about 4.0 mg per day for about 7 to about 14 days followed by about 14 to about 21 day of rest in a 28 day cycle combined with romidepsin administered intravenously in a dose of about 10 mg/m² to about 300 mg/m² administered on days 1, 8 and 15 of the 28 day cycle.

In one embodiment, 5-azacitidine is administered subcutaneously to patients with lymphoma in an amount of from about 0.10 to about 4.0 mg per day for about 7 to about 14 days followed by about 14 to about 21 day of rest in a 28 day cycle combined with romidepsin administered orally in a dose of about 10 mg/m² to about 300 mg/m² administered on days 1, 8 and 15 of the 28 day cycle.

In one embodiment, 5-azacitidine is administered orally to patients with lymphoma in an amount of from about 0.10 to about 4.0 mg per day for about 7 to about 14 days followed by about 14 to about 21 day of rest in a 28 day cycle combined with romidepsin administered orally in a dose of about 10 mg/m² to about 300 mg/m² administered on days 1, 8 and 15 of the 28 day cycle.

In one embodiment, 5-azacitidine and romidepsin are administered intravenously, with administration of romidepsin occurring 30 to 60 minutes prior to 5-azacitidine during a cycle of four to forty weeks. In another embodiment, 5-azacitidine is administered subcutaneously and romidepsin is administered by intravenous infusion. In another embodiment, 5-azacitidine is administered subcutaneously and romidepsin is administered orally. In yet another embodiment, 5-azacitidine and romidepsin are administered orally.

In one embodiment, 5-azacitidine and romidepsin are administered intravenously, with administration of 5-azacitidine occurring 30 to 60 minutes prior to romidepsin, during a cycle of four to forty weeks. In another embodiment, 5-azacitidine is administered subcutaneously and romidepsin is administered by intravenous infusion. In another embodiment, 5-azacitidine is administered subcutaneously and romidepsin is administered orally. In yet another embodiment, 5-azacitidine and romidepsin are administered orally.

In one embodiment, 5-azacitidine and romidepsin are administered intravenously, simultaneously, during a cycle of four to forty weeks. In another embodiment, 5-azacitidine is administered subcutaneously and romidepsin is administered by intravenous infusion. In another embodiment, 5-azacitidine is administered subcutaneously and romidepsin is administered orally. In yet another embodiment, 5-azacitidine and romidepsin are administered orally.

In one embodiment, one cycle comprises the administration of from about 0.1 to about 4.0 mg per day of 5-azacitidine and from about 25 to about 150 mg/m² of romidepsin daily for three to four weeks and then one or two weeks of rest. In one embodiment, the number of cycles during which the combinatorial treatment is administered to a patient will be from about one to about 40 cycles, or from about one to about 24 cycles, or from about two to about 16 cycles, or from about four to about three cycles.

Compositions

Romidepsin and 5-azacitidine can be used as compositions when combined with an acceptable carrier or excipient. Such compositions are useful in the methods provided herein.

Provided herein are pharmaceutical compositions comprising romidepsin as an active ingredient, including an enantiomer, a mixture of enantiomers, a mixture of two or more diastereomers, a tautomer, a mixture of two or more tautomers, or an isotopic variant thereof; or a pharmaceutically acceptable salt, solvate, hydrate, or prodrug in combination with a pharmaceutically acceptable vehicle, carrier, diluent, or excipient, or a mixture thereof.

Provided herein are pharmaceutical compositions comprising 5-azacitidine as an active ingredient or a pharmaceutically acceptable salt, solvate, hydrate, or prodrug in combination with a pharmaceutically acceptable vehicle, carrier, diluent, or excipient, or a mixture thereof.

Suitable excipients are well known to those skilled in the art, and non-limiting examples of suitable excipients are provided herein. Whether a particular excipient is suitable for incorporation into a pharmaceutical composition or dosage form depends on a variety of factors well known in the art, including, but not limited to, the method of administration. For example, oral dosage forms such as tablets may contain excipients not suited for use in parenteral dosage forms. The suitability of a particular excipient may also depend on the specific active ingredients in the dosage form. For example, the decomposition of some active ingredients may be accelerated by some excipients such as lactose, or when exposed to water. Active ingredients that comprise primary or secondary amines are particularly susceptible to such accelerated decomposition. Consequently, provided herein are pharmaceutical compositions and dosage forms that contain little, if any, lactose other mono- or disaccharides. As used herein, the term “lactose-free” means that the amount of lactose present, if any, is insufficient to substantially increase the degradation rate of an active ingredient. In one embodiment, lactose-free compositions comprise an active ingredient provided herein, a binder/filler, and a lubricant. In another embodiment, lactose-free dosage forms comprise an active ingredient, microcrystalline cellulose, pre-gelatinized starch, and magnesium stearate.

Like the amounts and types of excipients, the amounts and specific types of active ingredients in a dosage form may differ depending on factors such as, but not limited to, the route by which it is to be administered to patients. In one embodiment, dosage forms provided herein comprise romidepsin or a pharmaceutically acceptable salt, solvate, hydrate, stereoisomer, clathrate, or prodrug thereof in an amount of from about 0.5 mg/m² to 28 mg/m². In another embodiment, dosage forms provided herein comprise romidepsin or a pharmaceutically acceptable salt, solvate, hydrate, stereoisomer, clathrate, or prodrug thereof in an amount of about 8 mg/m², 10 mg/m², 12 mg/m², or 14 mg/m².

In one embodiment, dosage forms provided herein comprise 5-azacitidine or a pharmaceutically acceptable salt, solvate, hydrate, stereoisomer, clathrate, or prodrug thereof in an amount of from about 10 to about 150 mg/m². In another embodiment, dosage forms provided herein comprise 5-azacitidine or a pharmaceutically acceptable salt, solvate, hydrate, stereoisomer, clathrate, or prodrug thereof in an amount of about 10, 25, 50, 75, 100, 125, or 150 mg/m². In a specific embodiment, a dosage form comprises 5-azacitidine in an amount of about 50, 75 or 100 mg/m².

Pharmaceutical compositions provided herein can be used in the preparation of individual, single unit dosage forms. Single unit dosage forms are suitable for oral, mucosal (e.g., nasal, sublingual, vaginal, buccal, or rectal), parenteral (e.g., subcutaneous, intravenous, bolus injection, intramuscular, or intraarterial), topical (e.g., eye drops or other ophthalmic preparations), transdermal or transcutaneous administration to a patient. Examples of dosage forms include, but are not limited to: tablets; caplets; capsules, such as soft elastic gelatin capsules; cachets; troches; lozenges; dispersions; suppositories; powders; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; eye drops or other ophthalmic preparations suitable for topical administration; and sterile solids (e.g., crystalline or amorphous solids) that can be reconstituted to provide liquid dosage forms suitable for parenteral administration to a patient.

In one embodiment, the pharmaceutical compositions provided herein formulated in various dosage forms for oral administration.

In one embodiment, the pharmaceutical compositions provided herein formulated in various dosage forms for parenteral administration. In a specific embodiment, the pharmaceutical compositions provided herein formulated in various dosage forms for intravenous administration. In a specific embodiment, the pharmaceutical compositions provided herein formulated in various dosage forms for subcutaneous administration.

In one embodiment, the pharmaceutical compositions are provided in a dosage form for oral administration, which comprise romidepsin or a pharmaceutically acceptable salt, solvate, hydrate, or prodrug thereof; and one or more pharmaceutically acceptable excipients or carriers. In one embodiment, a dosage form is a capsule or tablet comprising romidepsin in an amount of about 10 mg/m², 25 mg/m², 50 mg/m², 100 mg/m², 200 mg/m², or 300 mg/m². In another embodiment, capsule or tablet dosage form comprises romidepsin in an amount of about 50 mg/m² or 75 mg/m².

In one embodiment, the pharmaceutical compositions are provided in a dosage form for parenteral administration, which comprise romidepsin or a pharmaceutically acceptable salt, solvate, hydrate, or prodrug thereof; and one or more pharmaceutically acceptable excipients or carriers. In one embodiment, a dosage form is a syringe or vial comprising romidepsin in an amount of about 0.5 mg/m², 2.5 mg/m², 7.5 mg/m², 15 mg/m², 20 mg/m², or 28 mg/m². In another embodiment, syringe or vial dosage form comprises romidepsin in an amount of about 8 mg/m², 10 mg/m², 12 mg/m², or 14 mg/m².

In one embodiment, the pharmaceutical compositions are provided in a dosage form for parenteral administration, which comprise 5-azacitidine or a pharmaceutically acceptable salt, solvate, hydrate, or prodrug thereof; and one or more pharmaceutically acceptable excipients or carriers. In one embodiment, a dosage form is a syringe or vial comprising 5-azacitidine in the amount of 10, 25, 50, 75, 100, 125, or 150 mg/m². In another embodiment, a syringe or vial dosage form comprises 5-azacitidine in an amount of about 50, 75, or 100 mg/m².

The pharmaceutical compositions provided herein can be provided in a unit-dosage form or multiple-dosage form. Examples of a unit-dosage form include an ampoule, syringe, and individually packaged tablet and capsule. For example, a 100 mg unit dose contains about 100 mg of an active ingredient in a packaged tablet or capsule. A unit-dosage form may be administered in fractions or multiples thereof. A multiple-dosage form is a plurality of identical unit-dosage forms packaged in a single container to be administered in segregated unit-dosage form. Examples of a multiple-dosage form include a vial, bottle of tablets or capsules, or bottle of pints or gallons.

The pharmaceutical compositions provided herein can be administered at once, or multiple times at intervals of time. It is understood that the precise dosage and duration of treatment may vary with the age, weight, and condition of the patient being treated, and may be determined empirically using known testing protocols or by extrapolation from in vivo or in vitro test or diagnostic data. It is further understood that for any particular individual, specific dosage regimens should be adjusted over time according to the individual need and the professional judgment of the person administering or supervising the administration of the formulations.

### A. Oral Administration

The pharmaceutical compositions provided herein for oral administration can be provided in solid, semisolid, or liquid dosage forms for oral administration. As used herein, oral administration also includes buccal, lingual, and sublingual administration. Suitable oral dosage forms include, but are not limited to, tablets, fastmelts, chewable tablets, capsules, pills, strips, troches, lozenges, pastilles, cachets, pellets, medicated chewing gum, bulk powders, effervescent or non-effervescent powders or granules, oral mists, solutions, emulsions, suspensions, wafers, sprinkles, elixirs, and syrups. In addition to the active ingredient(s), the pharmaceutical compositions can contain one or more pharmaceutically acceptable carriers or excipients, including, but not limited to, binders, fillers, diluents, disintegrants, wetting agents, lubricants, glidants, coloring agents, dye-migration inhibitors, sweetening agents, flavoring agents, emulsifying agents, suspending and dispersing agents, preservatives, solvents, non-aqueous liquids, organic acids, and sources of carbon dioxide.

Binders or granulators impart cohesiveness to a tablet to ensure the tablet remaining intact after compression. Suitable binders or granulators include, but are not limited to, starches, such as corn starch, potato starch, and pre-gelatinized starch (e.g., STARCH 1500); gelatin; sugars, such as sucrose, glucose, dextrose, molasses, and lactose; natural and synthetic gums, such as acacia, alginic acid, alginates, extract of Irish moss, panwar gum, ghatti gum, mucilage of isabgol husks, carboxymethylcellulose, methylcellulose, polyvinylpyrrolidone (PVP), Veegum, larch arabogalactan, powdered tragacanth, and guar gum; celluloses, such as ethyl cellulose, cellulose acetate, carboxymethyl cellulose calcium, sodium carboxymethyl cellulose, methyl cellulose, hydroxyethylcellulose (HEC), hydroxypropylcellulose (HPC), hydroxypropyl methyl cellulose (HPMC); microcrystalline celluloses, such as AVICEL-PH-101, AVICEL-PH-103, AVICEL RC-581, AVICEL-PH-105 (FMC Corp., Marcus Hook, Pa.); and mixtures thereof. Suitable fillers include, but are not limited to, talc, calcium carbonate, microcrystalline cellulose, powdered cellulose, dextrates, kaolin, mannitol, silicic acid, sorbitol, starch, pre-gelatinized starch, and mixtures thereof. The amount of a binder or filler in the pharmaceutical compositions provided herein varies upon the type of formulation, and is readily discernible to those of ordinary skill in the art. The binder or filler may be present from about 50 to about 99% by weight in the pharmaceutical compositions provided herein.

Suitable diluents include, but are not limited to, dicalcium phosphate, calcium sulfate, lactose, sorbitol, sucrose, inositol, cellulose, kaolin, mannitol, sodium chloride, dry starch, and powdered sugar. Certain diluents, such as mannitol, lactose, sorbitol, sucrose, and inositol, when present in sufficient quantity, can impart properties to some compressed tablets that permit disintegration in the mouth by chewing. Such compressed tablets can be used as chewable tablets. The amount of a diluent in the pharmaceutical compositions provided herein varies upon the type of formulation, and is readily discernible to those of ordinary skill in the art.

Suitable disintegrants include, but are not limited to, agar; bentonite; celluloses, such as methylcellulose and carboxymethylcellulose; wood products; natural sponge; cation-exchange resins; alginic acid; gums, such as guar gum and Veegum HV; citrus pulp; cross-linked celluloses, such as croscarmellose; cross-linked polymers, such as crospovidone; cross-linked starches; calcium carbonate; microcrystalline cellulose, such as sodium starch glycolate; polacrilin potassium; starches, such as corn starch, potato starch, tapioca starch, and pre-gelatinized starch; clays; aligns; and mixtures thereof. The amount of a disintegrant in the pharmaceutical compositions provided herein varies upon the type of formulation, and is readily discernible to those of ordinary skill in the art. The amount of a disintegrant in the pharmaceutical compositions provided herein varies upon the type of formulation, and is readily discernible to those of ordinary skill in the art. The pharmaceutical compositions provided herein may contain from about 0.5 to about 15% or from about 1 to about 5% by weight of a disintegrant.

Suitable lubricants include, but are not limited to, calcium stearate; magnesium stearate; mineral oil; light mineral oil; glycerin; sorbitol; mannitol; glycols, such as glycerol behenate and polyethylene glycol (PEG); stearic acid; sodium lauryl sulfate; talc; hydrogenated vegetable oil, including peanut oil, cottonseed oil, sunflower oil, sesame oil, olive oil, corn oil, and soybean oil; zinc stearate; ethyl oleate; ethyl laureate; agar; starch; lycopodium; silica or silica gels, such as AEROSIL® 200 (W.R. Grace Co., Baltimore, Md.) and CAB-O-SIL® (Cabot Co. of Boston, Mass.); and mixtures thereof. The pharmaceutical compositions provided herein may contain about 0.1 to about 5% by weight of a lubricant.

Suitable glidants include, but are not limited to, colloidal silicon dioxide, CAB-O-SIL® (Cabot Co. of Boston, Mass.), and asbestos-free talc. Suitable coloring agents include, but are not limited to, any of the approved, certified, water soluble FD&C dyes, and water insoluble FD&C dyes suspended on alumina hydrate, and color lakes and mixtures thereof. A color lake is the combination by adsorption of a water-soluble dye to a hydrous oxide of a heavy metal, resulting in an insoluble form of the dye. Suitable flavoring agents include, but are not limited to, natural flavors extracted from plants, such as fruits, and synthetic blends of compounds which produce a pleasant taste sensation, such as peppermint and methyl salicylate. Suitable sweetening agents include, but are not limited to, sucrose, lactose, mannitol, syrups, glycerin, and artificial sweeteners, such as saccharin and aspartame. Suitable emulsifying agents include, but are not limited to, gelatin, acacia, tragacanth, bentonite, and surfactants, such as polyoxyethylene sorbitan monooleate (TWEEN® 20), polyoxyethylene sorbitan monooleate 80 (TWEEN® 80), and triethanolamine oleate. Suitable suspending and dispersing agents include, but are not limited to, sodium carboxymethylcellulose, pectin, tragacanth, Veegum, acacia, sodium carbomethylcellulose, hydroxypropyl methylcellulose, and polyvinylpyrrolidone.

Suitable preservatives include, but are not limited to, glycerin, methyl and propylparaben, benzoic add, sodium benzoate and alcohol. Suitable wetting agents include, but are not limited to, propylene glycol monostearate, sorbitan monooleate, diethylene glycol monolaurate, and polyoxyethylene lauryl ether. Suitable solvents include, but are not limited to, glycerin, sorbitol, ethyl alcohol, and syrup. Suitable non-aqueous liquids utilized in emulsions include, but are not limited to, mineral oil and cottonseed oil. Suitable organic acids include, but are not limited to, citric and tartaric acid. Suitable sources of carbon dioxide include, but are not limited to, sodium bicarbonate and sodium carbonate.

It should be understood that many carriers and excipients may serve a plurality of functions, even within the same formulation.

The pharmaceutical compositions provided herein for oral administration can be provided as compressed tablets, tablet triturates, chewable lozenges, rapidly dissolving tablets, multiple compressed tablets, or enteric-coating tablets, sugar-coated, or film-coated tablets. Enteric-coated tablets are compressed tablets coated with substances that resist the action of stomach acid but dissolve or disintegrate in the intestine, thus protecting the active ingredients from the acidic environment of the stomach. Enteric-coatings include, but are not limited to, fatty acids, fats, phenyl salicylate, waxes, shellac, ammoniated shellac, and cellulose acetate phthalates. Sugar-coated tablets are compressed tablets surrounded by a sugar coating, which may be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. Film coating imparts the same general characteristics as sugar coating. Multiple compressed tablets are compressed tablets made by more than one compression cycle, including layered tablets, and press-coated or dry-coated tablets.

The tablet dosage forms can be prepared from the active ingredient in powdered, crystalline, or granular forms, alone or in combination with one or more carriers or excipients described herein, including binders, disintegrants, controlled-release polymers, lubricants, diluents, and/or colorants. Flavoring and sweetening agents are especially useful in the formation of chewable tablets and lozenges.

The pharmaceutical compositions provided herein for oral administration can be provided as soft or hard capsules, which can be made from gelatin, methylcellulose, starch, or calcium alginate. The hard gelatin capsule, also known as the dry-filled capsule (DFC), consists of two sections, one slipping over the other, thus completely enclosing the active ingredient. The soft elastic capsule (SEC) is a soft, globular shell, such as a gelatin shell, which is plasticized by the addition of glycerin, sorbitol, or a similar polyol. The soft gelatin shells may contain a preservative to prevent the growth of microorganisms. Suitable preservatives are those as described herein, including methyl- and propyl-parabens, and sorbic acid. The liquid, semisolid, and solid dosage forms provided herein may be encapsulated in a capsule. Suitable liquid and semisolid dosage forms include solutions and suspensions in propylene carbonate, vegetable oils, or triglycerides. Capsules containing such solutions can be prepared as described in U.S. Pat. Nos. 4,328,245; 4,409,239; and 4,410,545. The capsules may also be coated as known by those of skill in the art in order to modify or sustain dissolution of the active ingredient.

The pharmaceutical compositions provided herein for oral administration can be provided in liquid and semisolid dosage forms, including emulsions, solutions, suspensions, elixirs, and syrups. An emulsion is a two-phase system, in which one liquid is dispersed in the form of small globules throughout another liquid, which can be oil-in-water or water-in-oil. Emulsions may include a pharmaceutically acceptable non-aqueous liquid or solvent, emulsifying agent, and preservative. Suspensions may include a pharmaceutically acceptable suspending agent and preservative. Aqueous alcoholic solutions may include a pharmaceutically acceptable acetal, such as a di(lower alkyl)acetal of a lower alkyl aldehyde, e.g., acetaldehyde diethyl acetal; and a water-miscible solvent having one or more hydroxyl groups, such as propylene glycol and ethanol. Elixirs are clear, sweetened, and hydroalcoholic solutions. Syrups are concentrated aqueous solutions of a sugar, for example, sucrose, and may also contain a preservative. For a liquid dosage form, for example, a solution in a polyethylene glycol may be diluted with a sufficient quantity of a pharmaceutically acceptable liquid carrier, e.g., water, to be measured conveniently for administration.

Other useful liquid and semisolid dosage forms include, but are not limited to, those containing the active ingredient(s) provided herein, and a dialkylated mono- or poly-alkylene glycol, including, 1,2-dimethoxymethane, diglyme, triglyme, tetraglyme, polyethylene glycol-350-dimethyl ether, polyethylene glycol-550-dimethyl ether, polyethylene glycol-750-dimethyl ether, wherein 350, 550, and 750 refer to the approximate average molecular weight of the polyethylene glycol. These formulations can further comprise one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, bisulfite, sodium metabisulfite, thiodipropionic acid and its esters, and dithiocarbamates.

The pharmaceutical compositions provided herein for oral administration can be also provided in the forms of liposomes, micelles, microspheres, or nanosystems. Micellar dosage forms can be prepared as described in U.S. Pat. No. 6,350,458.

The pharmaceutical compositions provided herein for oral administration can be provided as non-effervescent or effervescent, granules and powders, to be reconstituted into a liquid dosage form. Pharmaceutically acceptable carriers and excipients used in the non-effervescent granules or powders may include diluents, sweeteners, and wetting agents. Pharmaceutically acceptable carriers and excipients used in the effervescent granules or powders may include organic acids and a source of carbon dioxide.

Coloring and flavoring agents can be used in all of the above dosage forms.

The pharmaceutical compositions provided herein for oral administration can be formulated as immediate or modified release dosage forms, including delayed-, sustained, pulsed-, controlled, targeted-, and programmed-release forms.

### B. Parenteral Administration

The pharmaceutical compositions provided herein can be administered parenterally by injection, infusion, or implantation, for local or systemic administration. Parenteral administration, as used herein, include intravenous, intraarterial, intraperitoneal, intrathecal, intraventricular, intraurethral, intrasternal, intracranial, intramuscular, intrasynovial, intravesical, and subcutaneous administration.

The pharmaceutical compositions provided herein for parenteral administration can be formulated in any dosage forms that are suitable for parenteral administration, including solutions, suspensions, emulsions, micelles, liposomes, microspheres, nanosystems, and solid forms suitable for solutions or suspensions in liquid prior to injection. Such dosage forms can be prepared according to conventional methods known to those skilled in the art of pharmaceutical science (see, Remington: The Science and Practice of Pharmacy, supra).

The pharmaceutical compositions intended for parenteral administration can include one or more pharmaceutically acceptable carriers and excipients, including, but not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, cryoprotectants, lyoprotectants, thickening agents, pH adjusting agents, and inert gases.

Suitable aqueous vehicles include, but are not limited to, water, saline, physiological saline or phosphate buffered saline (PBS), sodium chloride injection, Ringers injection, isotonic dextrose injection, sterile water injection, dextrose and lactated Ringers injection. Suitable non-aqueous vehicles include, but are not limited to, fixed oils of vegetable origin, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil, and palm seed oil. Suitable water-miscible vehicles include, but are not limited to, ethanol, 1,3-butanediol, liquid polyethylene glycol (e.g., polyethylene glycol 300 and polyethylene glycol 400), propylene glycol, glycerin, N-methyl-2-pyrrolidone, N,N-dimethylacetamide, and dimethyl sulfoxide.

Suitable antimicrobial agents or preservatives include, but are not limited to, phenols, cresols, mercurials, benzyl alcohol, chlorobutanol, methyl and propyl p-hydroxybenzoates, thimerosal, benzalkonium chloride (e.g., benzethonium chloride), methyl- and propyl-parabens, and sorbic acid. Suitable isotonic agents include, but are not limited to, sodium chloride, glycerin, and dextrose. Suitable buffering agents include, but are not limited to, phosphate and citrate. Suitable antioxidants are those as described herein, including bisulfite and sodium metabisulfite. Suitable local anesthetics include, but are not limited to, procaine hydrochloride. Suitable suspending and dispersing agents are those as described herein, including sodium carboxymethylcelluose, hydroxypropyl methylcellulose, and polyvinylpyrrolidone. Suitable emulsifying agents are those described herein, including polyoxyethylene sorbitan monolaurate, polyoxyethylene sorbitan monooleate 80, and triethanolamine oleate. Suitable sequestering or chelating agents include, but are not limited to EDTA. Suitable pH adjusting agents include, but are not limited to, sodium hydroxide, hydrochloric acid, citric acid, and lactic acid. Suitable complexing agents include, but are not limited to, cyclodextrins, including α-cyclodextrin, β-cyclodextrin, hydroxypropyl-β-cyclodextrin, sulfobutylether-β-cyclodextrin, and sulfobutylether 7-β-cyclodextrin (CAPTISOL®, CyDex, Lenexa, Kans.).

When the pharmaceutical compositions provided herein are formulated for multiple dosage administration, the multiple dosage parenteral formulations must contain an antimicrobial agent at bacteriostatic or fungistatic concentrations. All parenteral formulations must be sterile, as known and practiced in the art.

In one embodiment, the pharmaceutical compositions for parenteral administration are provided as ready-to-use sterile solutions. In another embodiment, the pharmaceutical compositions are provided as sterile dry soluble products, including lyophilized powders and hypodermic tablets, to be reconstituted with a vehicle prior to use. In yet another embodiment, the pharmaceutical compositions are provided as ready-to-use sterile suspensions. In yet another embodiment, the pharmaceutical compositions are provided as sterile dry insoluble products to be reconstituted with a vehicle prior to use. In still another embodiment, the pharmaceutical compositions are provided as ready-to-use sterile emulsions.

The pharmaceutical compositions provided herein for parenteral administration can be formulated as immediate or modified release dosage forms, including delayed-, sustained, pulsed-, controlled, targeted-, and programmed-release forms.

The pharmaceutical compositions provided herein for parenteral administration can be formulated as a suspension, solid, semi-solid, or thixotropic liquid, for administration as an implanted depot. In one embodiment, the pharmaceutical compositions provided herein are dispersed in a solid inner matrix, which is surrounded by an outer polymeric membrane that is insoluble in body fluids but allows the active ingredient in the pharmaceutical compositions diffuse through.

Suitable inner matrixes include, but are not limited to, polymethylmethacrylate, polybutyl-methacrylate, plasticized or unplasticized polyvinylchloride, plasticized nylon, plasticized polyethylene terephthalate, natural rubber, polyisoprene, polyisobutylene, polybutadiene, polyethylene, ethylene-vinyl acetate copolymers, silicone rubbers, polydimethylsiloxanes, silicone carbonate copolymers, hydrophilic polymers, such as hydrogels of esters of acrylic and methacrylic acid, collagen, cross-linked polyvinyl alcohol, and cross-linked partially hydrolyzed polyvinyl acetate.

Suitable outer polymeric membranes include but are not limited to, polyethylene, polypropylene, ethylene/propylene copolymers, ethylene/ethyl acrylate copolymers, ethylene/vinyl acetate copolymers, silicone rubbers, polydimethyl siloxanes, neoprene rubber, chlorinated polyethylene, polyvinylchloride, vinyl chloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer.

### C. Delayed Release Dosage Forms

Pharmaceutical compositions comprising romidepsin and 3-(4-amino-1-oxo-1,3-dihydro-isoindol-2-yl)-piperidine-2,6-dione can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled-release.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

Romidepsin Formulation

In one embodiment, romidepsin is formulated for injection as a sterile lyophilized white powder and is supplied in a single-use vial containing 10 mg romidepsin and 20 mg povidone, USP. The diluent is a sterile clear solution and is supplied in a single-use vial containing a 2 ml deliverable volume. The diluent for romidepsin contains 80% (v/v) propylene glycol, USP and 20% (v/v) dehydrated alcohol, USP. Romidepsin is supplied as a kit containing two vials.

Romidepsin for injection is intended for intravenous infusion after reconstitution with the supplied Diluent and after further dilution with 0.9% Sodium Chloride, USP.

5-azacitidine Formulation

In one embodiment, 5-azacitidine is formulated for injection as a sterile lyophilized powder and is supplied in a single-use vial containing 100 mg of 5-azacitidine and 100 mg of mannitol.

5-azacitidine for injection is intended for intravenous injection after reconstitution as a solution with further dilution. 5-azacitidine for injection is intended for subcutaneous injection after reconstitution as a suspension.

Kits

In one embodiment, provided herein are kits comprising one or more containers filled with romidepsin or a pharmaceutical composition thereof, and one or more containers filled with 5-azacitidine or a pharmaceutical composition thereof.

### EXAMPLES

### Example 1

### The Effect of the Combination of Romidepsin and 5-azacitidine On Apoptosis and Cell Viability in CTCL Cell Lines

Human CTCL cell lines (Hut78, SeAx, and MyLa) were grown in RPMI 1640 medium supplemented with 10% heat-inactivated fetal bovine serume (FBS), glutamine (2 mM) and streptomycin (100 μg/ml) at 37° C., 5% CO₂ and 95% humidity.

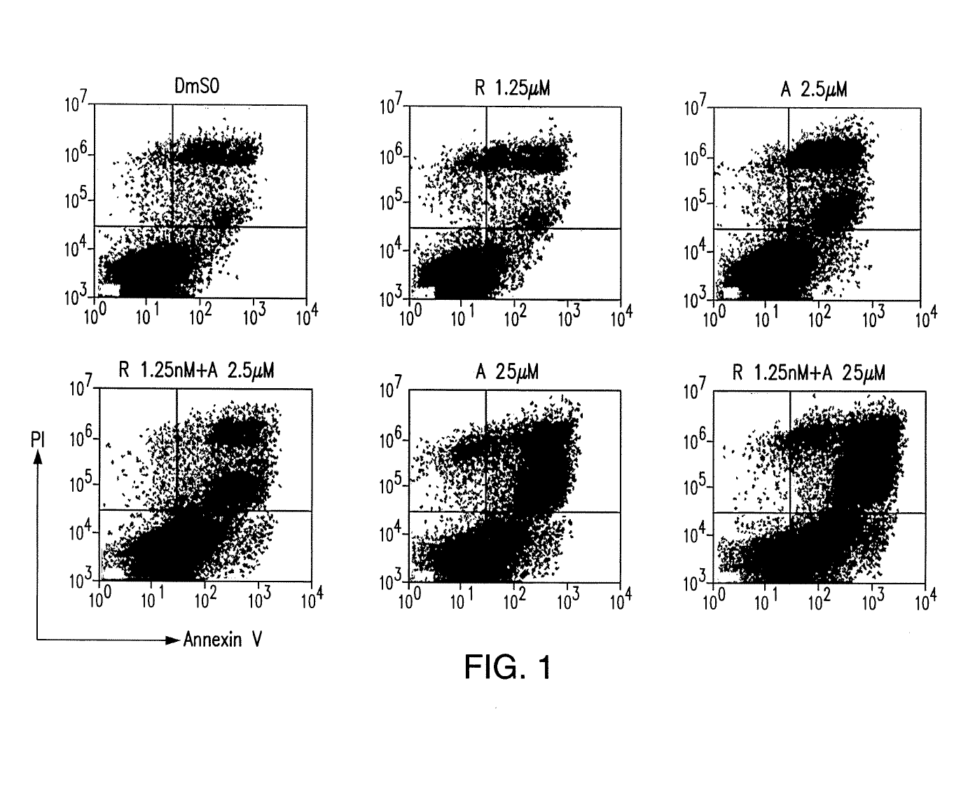

The CTCL primary cell lines cultures were treated with the following combinations of the drugs: each agent separately; sequential treatment with both drugs with a clearance period of 48 hours in between; and simultaneous treatment with both drugs. Romidepsin was used in a concentration ranging from 0.25 nM to 10 μM. 5-azacitidine was used in a concentration ranging from 500 nM to 10 μM. After 24 to 72 hours of the combined drug treatment, the levels of apoptosis and necrosis were measured by flow cytometry with Annexin V-FITC detection kit. The results are shown in FIGS. 1, 4A, 4B, and 5A-5D.

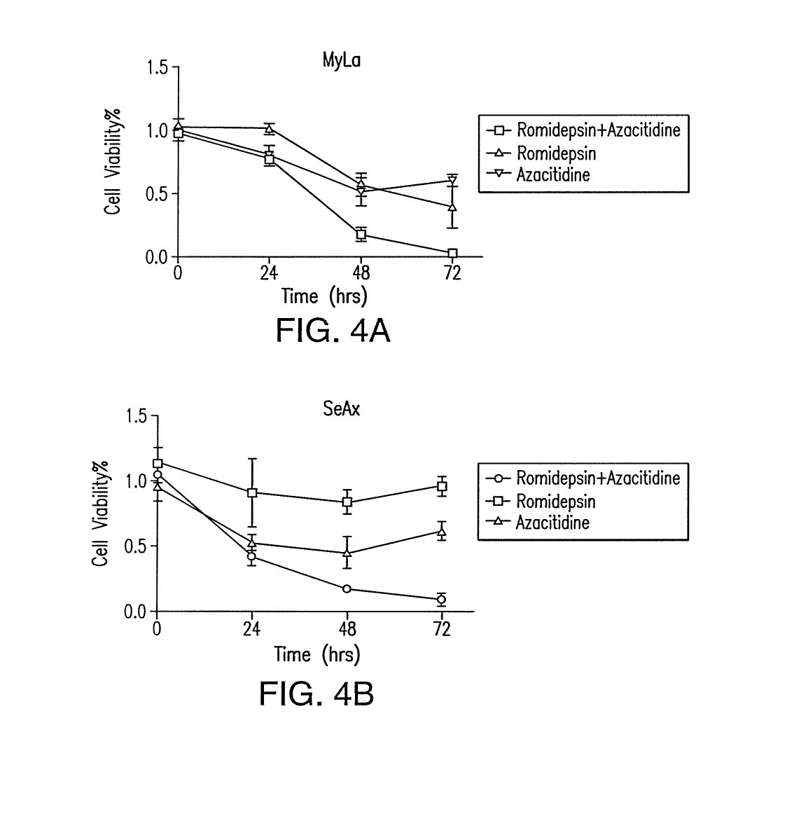

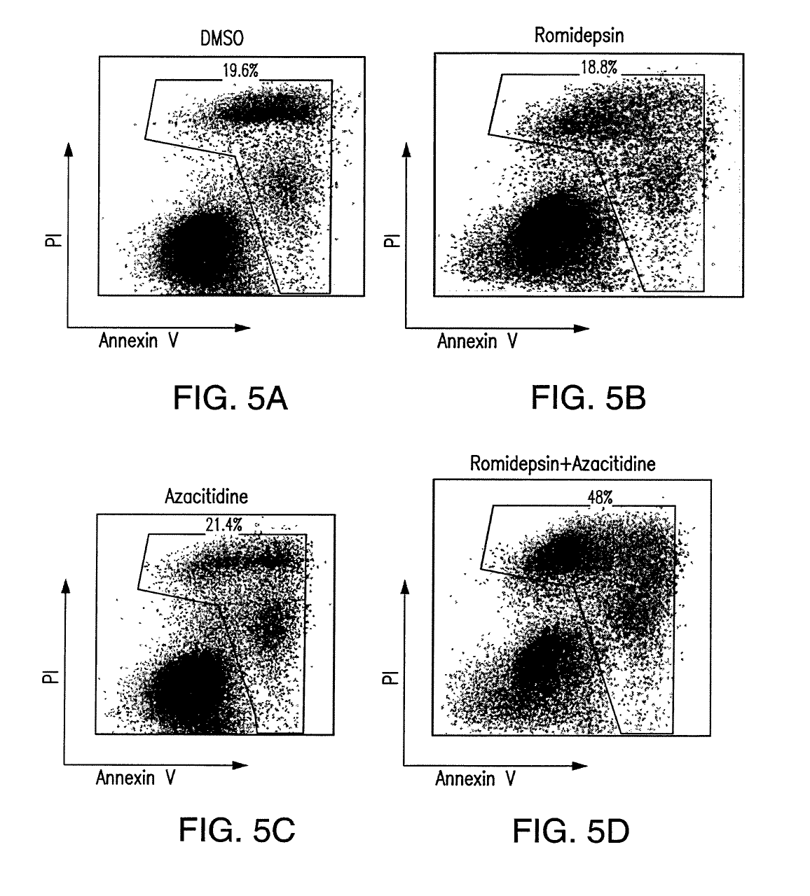

The combined treatment of the CTCL primary cell cultures with romidepsin and 5-azacitidine demonstrated a noticeable change of cell viability due to an increase in necrosis and apoptosis in a time and dose dependent manner (FIG. 1). Cell viability decreased significantly more after 48 hours of treatment with the combination of romidepsin and 5-azacitidine, compared to treatment with the individual agents. Almost complete cell death was seen after 72 hours treatment with the combined agents of the MyLa CTCL cell line (FIG. 4A), and the SeAx CTCL cell line (FIG. 4B). It was also shown that the combination of romidepsin and 5-azacitidine had synergistic effects on apoptosis in CTCL cells. The combined treatment resulted in 48% apoptosis, whereas the individual treatments resulted in 18.8% apoptosis for romidepsin and 21.4% for 5-azacitidine (FIGS. 5A-5D).

### Example 2

### The Effect of the Combination of Romidepsin and 5-azacitidine on Protein Expression of Cell Cycle Regulatory Genes

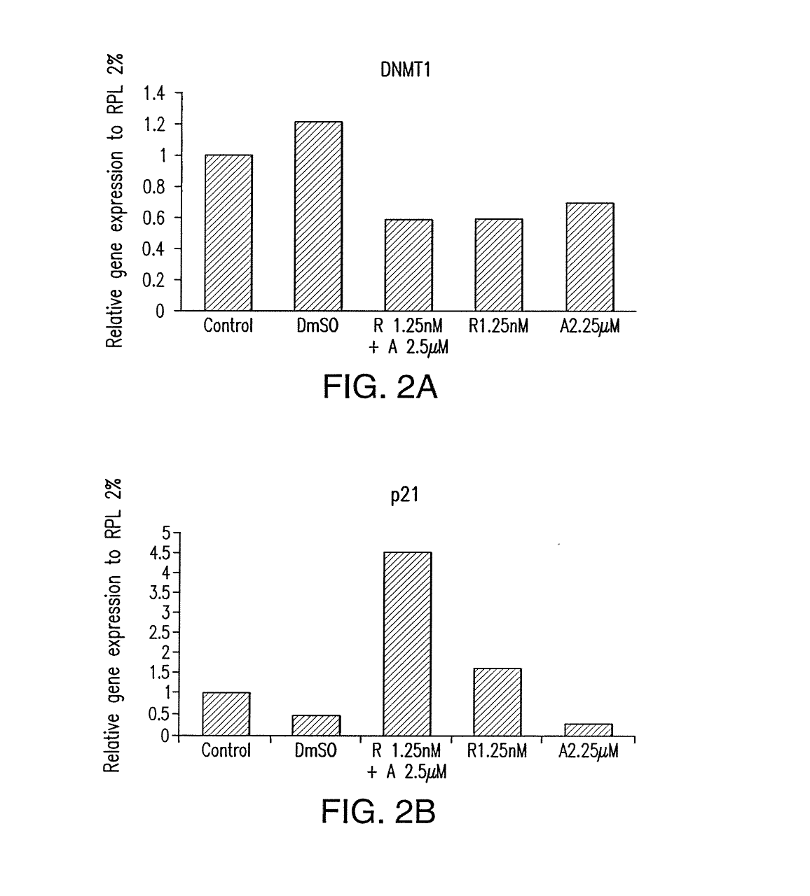

The gene expression levels of cell cycle regulatory genes (p15, p16, p21 and p27), HDACs (HDAC1, HDAC2, HDAC3, and HDAC6) and DNA methyltransferases (DNMT1, DNMT3a and DNMT3b) were examined by RT-PCR and immunohistochemical methods. The results are shown in FIGS. 2, 3, and 6-8.

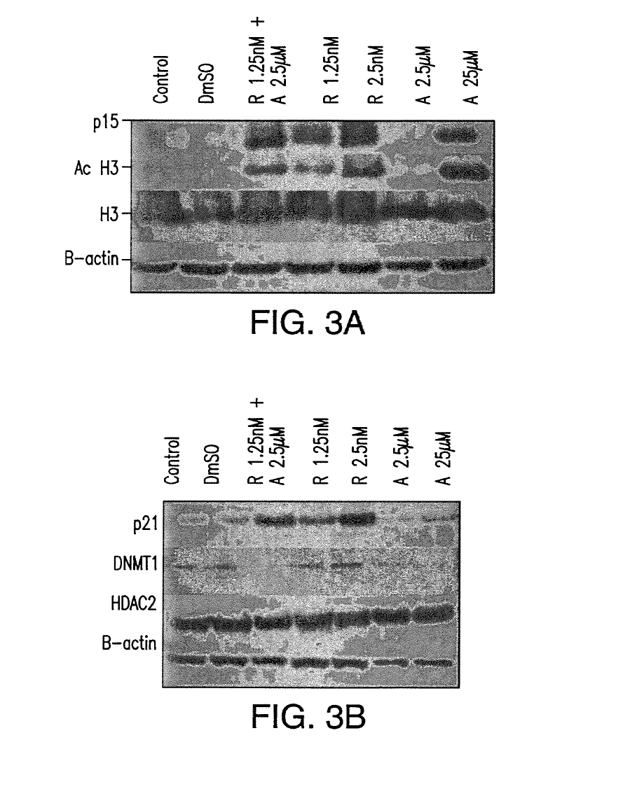

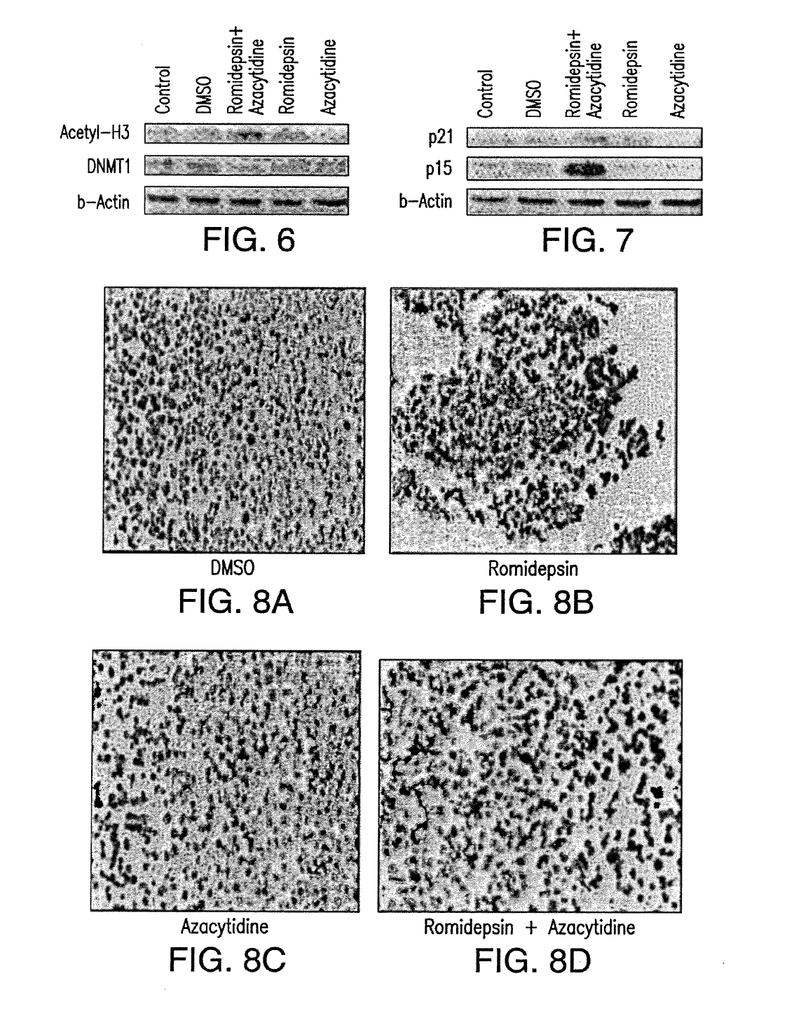

The combined use of romidepsin and 5-azacitidine resulted in significantly higher levels of expression of cell cycle regulatory genes like p21 (FIG. 2), p15 (FIGS. 3 and 7), and p16 (FIGS. 8 A-D), and in an increase in acetylation of H3 (FIG. 6). These findings suggest that the combination of romidepsin and 5-azacitidine induces cell cycle arrest and is able to stop the loss of cell cycle control in a more pronounce fashion than the single agents.

### Example 3

### The Effect of the Combination of Romidepsin and 5-azacitidine on Apoptotic Caspase Pathway

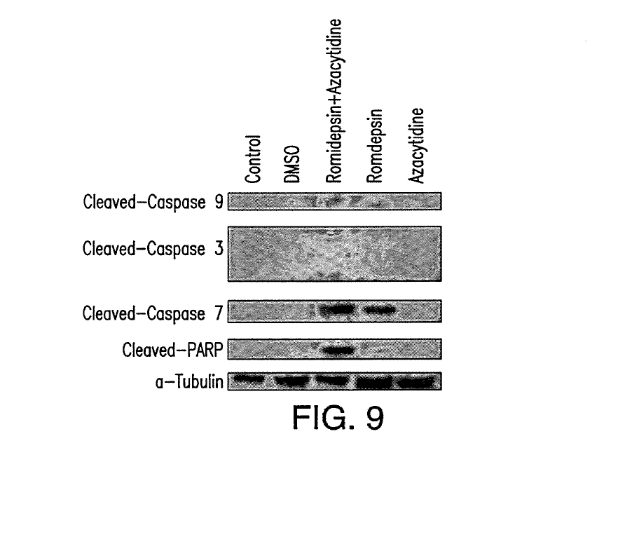

The expression levels of various caspases involved in an apoptosis-regulating cascade were determined by Western blot. The results are shown in FIG. 9.

The combination of romidepsin and 5-azacitidine demonstrated an increased cleavage of caspases 3, 7, and 9. These findings indicate that the synergistic effect demonstrated by the combination is based on the involvement of common (cleaved caspases 3 and 7) and intrinsic (cleaved caspase 9) apoptotic pathways.

Therefore, the combined treatment of the CTCL primary cell cultures with romidepsin and 5-azacitidine demonstrated promising results for use of this combination in patients with CTCL.

All publications, patents, and patent applications mentioned in this specification are herein incorporated by reference to the same extent as if each individual publication, patent, or patent application was specifically and individually indicated to be incorporated by reference.

The present disclosure has been described above with reference to exemplary embodiments. However, those skilled in the art, having read this disclosure, will recognize that changes and modifications may be made to the exemplary embodiments without departing from the scope of the present disclosure. The changes or modifications are intended to be included within the scope of the present disclosure, as expressed in the following claims.

What is claimed:

1. A method of treating lymphoma, said method comprising administering to a patient in need of such treatment a therapeutically effective amount of an HDAC inhibitor and a therapeutically effective amount of a DNA demethylating agent.

2. The method of claim 1, wherein the lymphoma is T-cell lymphoma.

3. The method of claim 2, wherein the T-cell lymphoma is relapsed, refractory or resistant to conventional therapy.

4. The method of claim 3, wherein the T-cell lymphoma is cutaneous T-cell lymphoma (CTCL).

5. The method of claim 4, wherein CTCL is refractory or relapsed CTCL.

6. The method of claim 1, wherein the HDAC inhibitor is romidepsin.

7. The method of claim 1, wherein the DNA demethylating agent is 5-azacitidine.

8. The method of claim 1, wherein the HDAC inhibitor is romidepsin in an amount of from about 0.5 to about 28 mg/m² per day and the DNA demethylating agent is 5-azacitidine in an amount of from about 10 to 150 mg/m² per day.

9. The method of claim 8, wherein 5-azacitidine and romidepsin are administered intravenously.

10. The method of claim 8, wherein the amount of 5-azacitidine is about 50, 75 or 100 mg/m² per day.

11. The method of claim 8, wherein the amount of romidepsin is about 8, 10, 12 or 14 mg/m² per day.

12. The method of claim 1, wherein the HDAC inhibitor is romidepsin in an amount of from about 10 to about 300 mg/m² per day and the DNA demethylating agent is 5-azacitidine in an amount of from about 10 to 150 mg/m² per day.

13. The method of claim 12, wherein 5-azacitidine is administered intravenously and romidepsin is administered orally.

14. The method of claim 12, wherein 5-azacitidine is administered subcutaneously and romidepsin is administered orally.

15. The method of claim 12, wherein 5-azacitidine is administered orally and romidepsin is administered intravenously.

16. The method of claim 12, wherein 5-azacitidine and romidepsin are administered orally.

17. The method of claim 12, wherein the amount of romidepsin is from about 25 to about 200 mg/m² per day.

18. The method of claim 12, wherein the amount of romidepsin administered is about 50, 75 or 100 mg/m² per day.

19. The method of claim 12, wherein the amount of 5-azacitidine is about 50, 75 or 100 mg/m² per day.

20. The method of claim 12, wherein 5-azacitidine is administered in an amount of about 50, 75 or 100 mg/m² per day for about 7 to about 14 days followed by about 21 to about 14 days rest in a 28 day cycle, and wherein romidepsin is administered in an amount of about 10 or 12 mg/m² per day on days 1, 8 and 15 of the 28 day cycle.
